(19) United States
(12) Patent Application Publication
(10) Pub. No.: US 2026/0054004 A1
(43) Pub. Date: Feb. 26, 2026

(54) CHEMICAL - LIQUID INJECTOR

(71) Applicant: NEMOTO KYORINDO CO., LTD., Tokyo (JP)

(72) Inventors: Shigeru NEMOTO, Tokyo (JP); Yumiko FUKIKOSHI, Tokyo (JP); Toshio KANETAKA, Tokyo (JP)

(21) Appl. No.: 18/854,717

(22) PCT Filed: Apr. 13, 2023
(86) PCT No.: PCT/JP2023/015097

§ 371 Date: May 29, 2025

(30) Foreign Application Priority Data
Apr. 14, 2022 (JP) 2022-067253

(51) Int. Cl. A61M 5/00; A61M 5/168

(52) U.S. Cl. CPC A61M 5/007; A61M 5/16813; A61M 5/16827; A61M 2205/505

(57) ABSTRACT

A chemical-liquid injector which is capable of controlling the flow of a chemical liquid in a chemical-liquid circuit. The chemical-liquid injector has a chemical-liquid circuit mounting section on which a chemical-liquid circuit having a patient line and a transducer line which is branched from the patient line as the flow channels of the chemical liquid, is mounted. The chemical liquid injector has a flattening mechanism on a downstream side of a branching portion of the patient line and the transducer line of the chemical-liquid circuit mounting section.

### TECHNICAL FIELD

The present invention relates to a chemical-liquid injector having a chemical-liquid circuit which includes a plurality of tubes.

### BACKGROUND ART

A chemical-liquid injector is largely used for injecting a chemical liquid into a patient. A large number of chemical-liquid injectors, from a viewpoint of the ease of injecting at a desired injection rate, have a detachably loaded syringe, and are configured to inject a chemical liquid filled into the syringe via a chemical-liquid circuit which fluidically connects the syringe and the patient.

The syringe, although essentially, is not used for more than once, a chemical liquid is refilled from a bottle into the syringe, and is often used for a plurality of times. In that case, the chemical-liquid circuit has a patient line leading from the syringe to the patient, and a bottle line leading from the patient line to the bottle upon branching. Although the bottle line is closed at the time of injecting the chemical liquid, at the time of filling the chemical liquid, the bottle line is opened after the patient line is closed at a downstream side of a branching portion of the bottle line. At this time, it is significant to make an arrangement such that, blood of the patient that regurgitates through the patient line (reverse blood) does not reach an upstream side of the closing portion of the patient line.

As a chemical-liquid circuit which is capable of preventing such reverse flow of blood more reliably, a chemical-liquid circuit which is configured such that the closing portion includes a first moving member having a flow channel, a second moving member which has a flow channel and which is positioned on a downstream side of the patient line of the first moving member, and a housing which slidably accommodates the first moving member and the second moving member, wherein the flow channel of the second moving member opens after the flow channel of the first moving member has opened, has been disclosed in Patent Literature 1 (International Unexamined Patent Application Publication No. 2018/181270) for example.

### PRIOR ART DOCUMENT

### Patent Literature

Patent Literature 1: International Unexamined Patent Application Publication No. 2018/181270

### SUMMARY OF THE INVENTION

### Problems to be Solved by the Invention

The chemical-liquid circuit, for controlling the flow of a chemical liquid in accordance with an operation of the chemical-liquid injector, has various components such as a flow channel opening/closing valve, besides tubes. One of the objects of the present invention is to provide a chemical-liquid injector which is capable of controlling satisfactorily the flow of a chemical liquid in the chemical-liquid circuit having various components.

### Means for Solving the Problems

According to an aspect of the present invention, there is provided a chemical-liquid injector on which a chemical-liquid circuit is detachably mounted, which includes
a chemical-liquid circuit mounting section on which the chemical-liquid circuit having a patient line and a transducer line which is branched from the patient line, as flow channels of the chemical liquid, and
a flow channel opening/closing mechanism which is at a downstream side of a branching portion of the patient line and the transducer line, of the chemical-liquid circuit mounting section,

### (Definition of Terms)

In the present specification, ‘upstream’ and ‘downstream’ signify ‘upstream’ and ‘downstream’ with respect to a direction of flow of a chemical liquid. However, in a case in which the chemical liquid can flow in both directions of injecting and sucking of the chemical liquid, the terms signify ‘upstream’ and ‘downstream’ with respect to a direction of flow of the chemical liquid at the time of injecting the chemical liquid, unless specifically noted.

### Effects of the Invention

According to the present invention, it is possible to provide a chemical-liquid injector which is capable of controlling satisfactorily the flow of a chemical liquid in a chemical-liquid circuit.

### BRIEF DESCRIPTION OF THE DRAWINGS

FIG. 1 is a schematic diagram of a medical imaging system according to an embodiment of the present invention.

FIG. 2 is a schematic diagram of a chemical-liquid circuit shown in FIG. 1.

FIG. 3 is an exploded perspective view of an embodiment of a syringe that can be used in a chemical-liquid injector shown in FIG. 1.

FIG. 3A is an exploded perspective view when another embodiment of a protective cover 21 is viewed from a rear side, shown together with a syringe which can be combined therewith.

FIG. 3B is a plan view of a tail end portion of the protective cover shown in FIG. 3A.

FIG. 3C is a linear cross-sectional view along a line 3C-3C of the protective cover shown in FIG. 3B.

FIG. 3D is a front view of an embodiment of an anti-slip ring that can be provided to an inner surface of the protective cover.

FIG. 3E is a perspective view of a syringe inserted into the protective cover, as viewed from a lower rear side.

FIG. 4 is a perspective view of an embodiment of a flow channel opening/closing valve shown in FIG. 2.

FIG. 5 is a perspective view of a housing of the flow channel opening/closing valve shown in FIG. 4.

FIG. 5A is a linear cross-sectional perspective view along a line 5A-5A of the housing shown in FIG. 5.

FIG. 6 is a perspective view of a piston of the flow channel opening/closing valve shown in FIG. 4.

FIG. 6A is a linear cross-sectional perspective view along a line 6A-6A of the piston shown in FIG. 6.

FIG. 6B is a linear cross-sectional perspective view along a line 6B-6B of the piston shown in FIG. 6.

FIG. 7A is a linear-cross-sectional view along a line 7A-7A at an open position and a closed position of the flow channel opening/closing valve shown in FIG. 4.

FIG. 7B is a linear cross-sectional view along a line 7B-7B at the open position and the closed position of the flow channel opening/closing value shown in FIG. 4.

FIG. 7C is a cross-sectional view similar to FIG. 7B, showing an enlarged stopper mechanism shown in FIG. 7B.

FIG. 8A is a perspective view of an embodiment 1 of a unidirectional valve that can be used in a chemical-liquid circuit.

FIG. 8B is an exploded perspective view of the unidirectional valve shown in FIG. 8A.

FIG. 8C is a linear cross-sectional view along a line 8C-8C of the unidirectional valve shown in FIG. 8A.

FIG. 8D is a perspective view of a first case of the unidirectional valve shown in FIG. 8A.

FIG. 8E is a perspective view of a second case of the unidirectional valve shown in FIG. 8A.

FIG. 9A is a cross-sectional view showing a closed state of an embodiment 2 of the unidirectional valve that is used in a chemical-liquid circuit.

FIG. 9B is a cross-sectional view showing an open state of the embodiment 2 of the unidirectional valve that can be used in a chemical-liquid circuit.

FIG. 10A is a perspective view of an embodiment 3 of a unidirectional valve that can be used in a chemical-liquid circuit.

FIG. 10B is an exploded perspective view of the unidirectional valve shown in FIG. 10A.

FIG. 10C is a cross-sectional view of the unidirectional valve shown in FIG. 10A.

FIG. 11A is a perspective view of an embodiment 4 of a unidirectional valve that can be used in the chemical-liquid circuit.

FIG. 11B is an exploded perspective view of the unidirectional valve shown in FIG. 11A.

FIG. 11C is a cross-sectional view of the unidirectional valve shown in FIG. 11A.

FIG. 12 is a perspective view of a suction-tube unit that can be used in a chemical-liquid circuit.

FIG. 12A is a perspective view of a suction valve of the suction-tube unit shown in FIG. 12.

FIG. 12B is an exploded perspective view of the suction valve shown in FIG. 12A.

FIG. 12C is a linear cross-sectional perspective view along a line 12C-12C of the suction valve shown in FIG. 12A.

FIG. 12D is a perspective view of a spike of the suction-tube unit shown in FIG. 12A.

FIG. 12E is a linear cross-sectional perspective view along a line 12E-12E of the spike shown in FIG. 12A.

FIG. 13 is a side view in which a portion of a modified example of a syringe connector and a syringe is shown in a cross-section.

FIG. 14 is a block diagram showing a configuration of an injection head of the medical imaging system shown in FIG. 1.

FIG. 15 is a perspective view of a head body of the injection head shown in FIG. 14.

FIG. 15A is a diagram showing an open position of a clamper shown in FIG. 15.

FIG. 15B is a diagram showing a closed position of the clamper shown in FIG. 15.

FIG. 15C is a perspective view of a presser showing a relationship of a syringe and a plunger.

FIG. 15D is a perspective view when the presser is viewed from a rear upper side (in a direction of arrow FIG. 15D of FIG. 15C).

FIG. 15E is a plan view of a presser in a state in which a protrusion of the syringe is engaged.

FIG. 15F is a perspective view of an embodiment of a syringe drive mechanism.

FIG. 15G is a diagram showing schematically the configuration of a side frame, a ram, and a linear guide when the syringe drive mechanism is viewed from a front side.

FIG. 16 is a perspective view of another embodiment of an injection head.

FIG. 16A is a perspective view of the injection head shown in FIG. 16, in which a bottle is held in a first chemical-liquid container holder, and a large-capacity bag is held in a second chemical-liquid container holder.

FIG. 16B is a perspective view of the injection head shown in FIG. 16, in which the bottle is held in the first chemical-liquid container holder and the large-capacity bag is held in the second chemical-liquid container holder.

FIG. 16C is a perspective view of an upward posture of the injection head shown in FIG. 16.

FIG. 17 is a block diagram of an embodiment of a chemical-liquid injector having two consoles.

FIG. 18 is a perspective view of an embodiment of a variable hand switch shown in FIG. 17.

FIG. 19A is a diagram showing an embodiment of a screen displayed on a display device of a chemical-liquid injector.

FIG. 19B is a diagram showing an embodiment of a screen displayed on the display device of the chemical-liquid injector.

FIG. 19C is a diagram showing an embodiment of a screen displayed on the display device of the chemical-liquid injector.

FIG. 19D is a diagram showing an embodiment of a screen displayed on the display device of the chemical-liquid injector.

FIG. 19E is a diagram showing an embodiment of a screen displayed on the display device of the chemical-liquid injector.

FIG. 19F is a diagram showing an embodiment of a screen displayed on the display device of the chemical-liquid injector.

FIG. 19G is a diagram showing an embodiment of a screen displayed on the display device of the chemical-liquid injector.

FIG. 19H is a diagram showing an embodiment of a screen displayed on the display device of the chemical-liquid injector.

### EMBODIMENTS FOR CARRYING OUT THE INVENTION

Embodiments of the present invention will be described below by referring to the accompanying diagrams. Here, the description will be made by citing an example of an angio imaging system which is favorably used in cardiac catheter examination by coronary arteriography. However, the present invention is not restricted to this, and is also applicable to CT (Computed Tomography) imaging system, MRI (Magnetic Resonance Imaging) system, PETS (Positron Emission Tomography) system and the like.

### [A] Overall Configuration

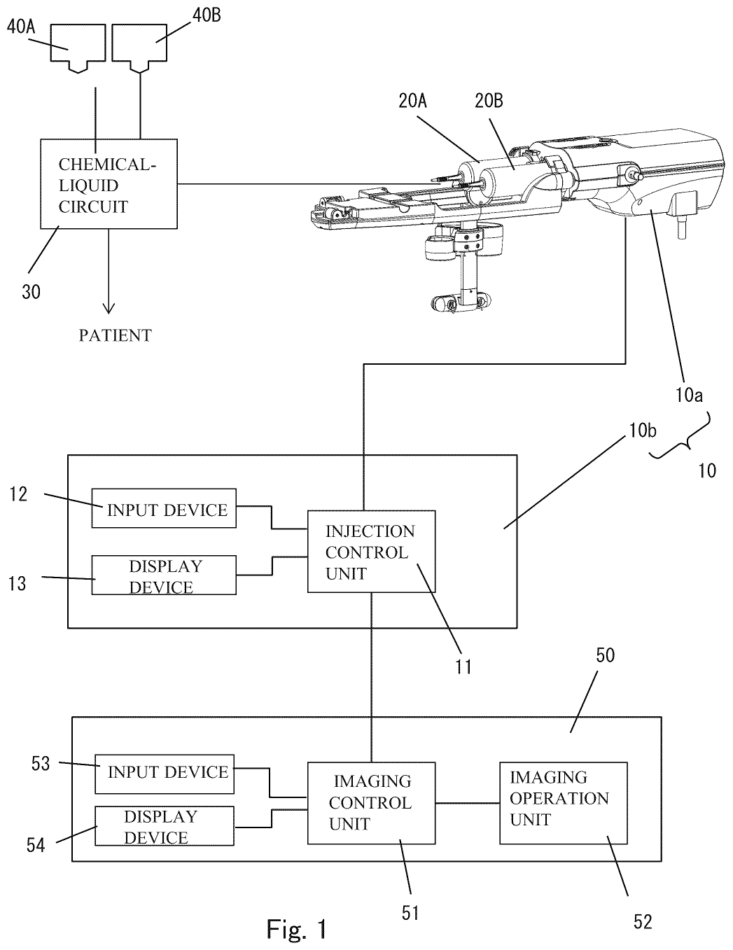

Referring to FIG. 1, a schematic diagram of a medical imaging system according to an embodiment of the present invention having a chemical-liquid injector 10, a chemical-liquid circuit 30, and a medical imaging apparatus 50 is shown. The chemical-liquid injector 10 has an injection head 10a and a console 10b. The chemical-liquid circuit 30 fluidically connects the injection head 10a and a patient. The chemical-liquid injector 10 and the medical imaging apparatus 50 can be mutually connected so as to carry out transmission and reception of data between each other. Connection between the two may be a wired connection or a wireless connection.

The medical imaging apparatus 50 has an imaging operation unit 52 which carries out an imaging operation and an imaging control unit 51 which controls the operation of the imaging operation unit 52, and is capable of acquiring a medical image including a tomographic image and/or three-dimensional image of the patient injected with a chemical liquid by the chemical-liquid injector 10. The imaging operation unit 52, normally, has an electromagnetic wave irradiation unit which irradiates electromagnetic waves to a bed for the patient and a predetermined space on the bed. The imaging control unit 51 controls an operation of the overall medical imaging apparatus such as. determining imaging conditions and controlling the operation of the imaging operation unit 52 according to the imaging conditions determined. It is possible to configure the imaging control unit 51 by including a so-called microcomputer, and can have a CPU (central processing unit), a ROM (read only memory), a RAM (random access memory), and an interface with other instruments. A computer program for a control of the medical imaging apparatus 50 is installed in the ROM. The CPU, by executing various functions according to the computer program, controls an operation of each section of the medical imaging apparatus 50.

The medical imaging apparatus 50 can further include a display device 54 such as a liquid crystal display which is capable of displaying imaging conditions and a medical image acquired, and an input device 53 for inputting imaging conditions etc. As the input device 53, it is possible to use at least one type of known input devices such as various buttons, a keyboard, and a mouse. At least a part of data to be used for determining the imaging conditions is input from the input device 53, and is transmitted to the imaging control unit 51. Data to be displayed on the display device 54 is transmitted from the imaging control unit 51. Moreover, it is possible to use a touch panel in which a touch screen is disposed as an input device on a display which is a display device, as the input device 53 and the display device 54. It is possible to incorporate a part of the input device 53, the display device 54, and the imaging control unit 51 in one housing as a console for a medical imaging apparatus.

The chemical-liquid injector 10 is an apparatus used for injecting a chemical liquid filled in a syringe into a blood vessel of a patient via the chemical-liquid circuit 30. The syringe is detachably loaded in the injection head 10a, and at least one syringe drive mechanism which operates a plunger (or a piston) of the syringe is built-in in the injection head 10a. In the present embodiment, the injection head 10a is configured such that it is possible to load two syringes 20A and 20B in order to be able to inject separately or simultaneously two types of chemical liquids such as a contrast medium and a physiological saline solution for example, and moreover, has two syringe drive mechanisms which operate each of the syringes 20A and 20B independently. However, at least one of the syringe drive mechanism for injecting one chemical liquid and the syringe drive mechanism for injecting the other chemical liquid may be in plurality.

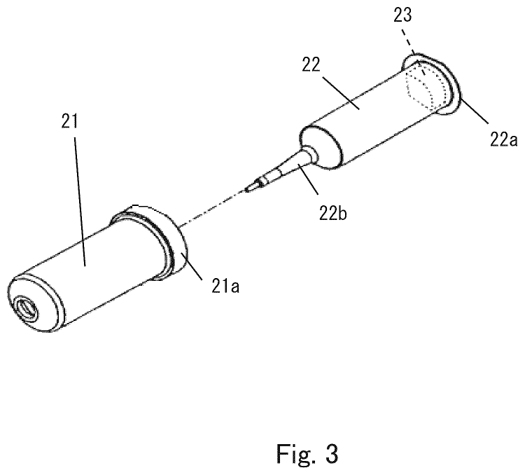

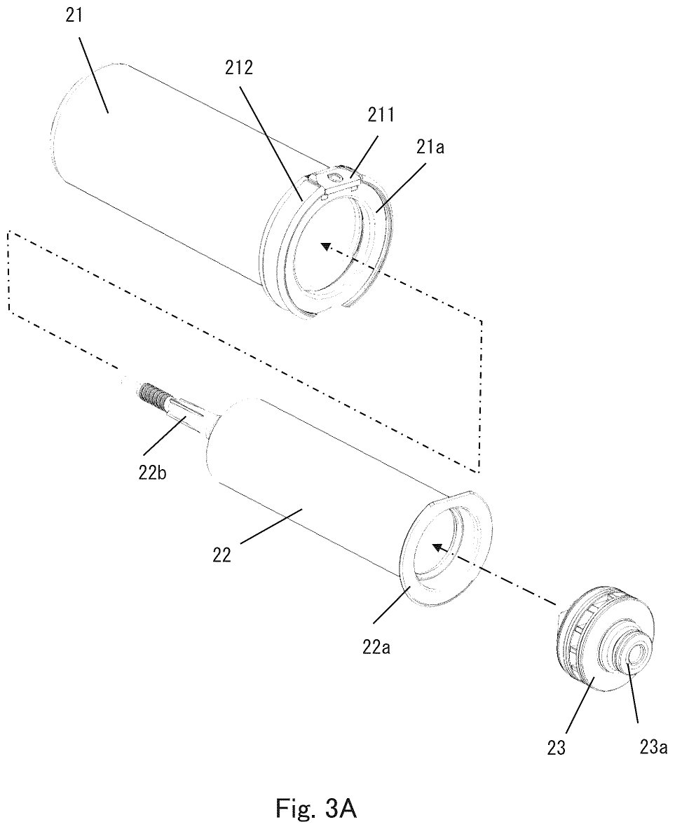

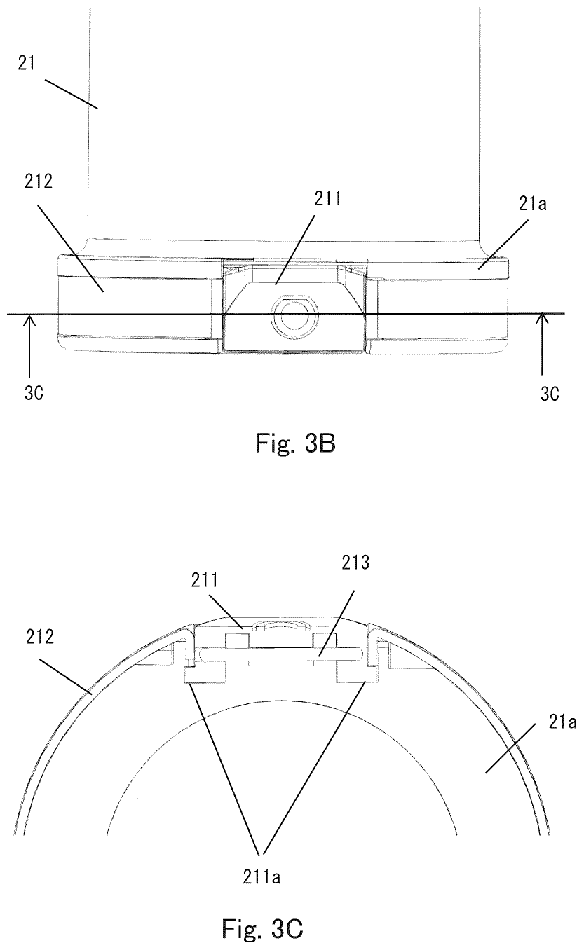

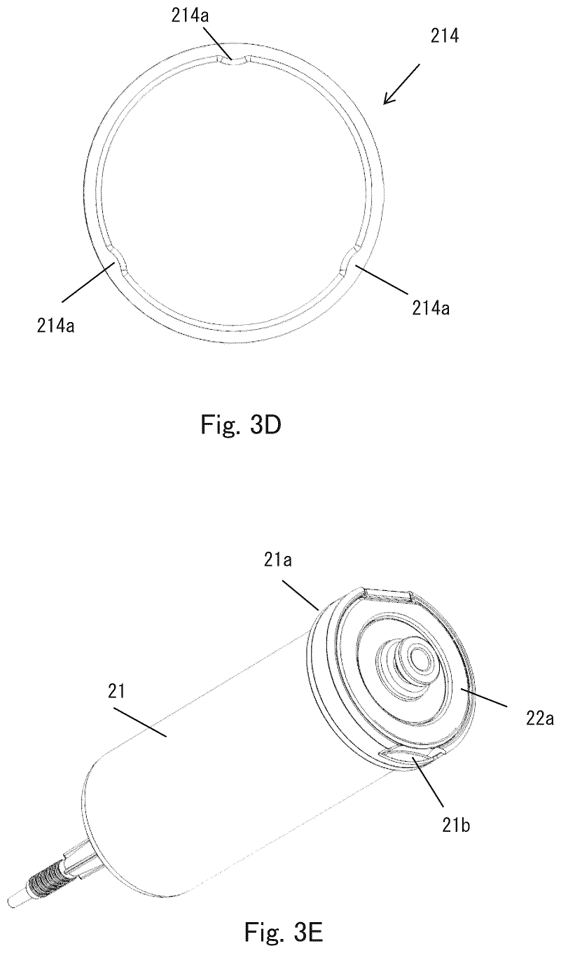

Here, an embodiment of a syringe that can be used in the present embodiment will be described below by referring to FIG. 3. The syringe shown in the diagram is a syringe normally called as a rod less syringe, and has a cylinder 22 having a flange 22a and a nozzle portion 22b formed at a tail end and a front end respectively, and a plunger 23 which is inserted to be movable back and forth in the cylinder 22. At a tail end of the plunger 23, a projection (not shown in the diagram) in the form of a flange to be engaged with the cylinder drive mechanism of the injection head 10a is formed integrally. The syringe may be a syringe of a pre-filled type which is provided by a manufacturer in a state of a chemical liquid filled therein, or may be a syringe of a type to be filled on site having a chemical liquid filled at a medical site.

The syringe is inserted into a protective cover 21, and is loaded in the injection head 10a. The protective cover 21, in order to suppress an expansion by a rise in internal pressure of the cylinder 22 during the chemical liquid injection, is a component configured to be circular cylindrical shaped having dimensions such that there is not gap practically between an outer peripheral surface of the cylinder 22 and an inner peripheral surface of the protective cover 21. In order that the protective cover 21 carries out this function, the protective cover 21 is formed to be thick-walled having a mechanical strength that can adequately withstand the internal pressure acting on the cylinder 22 during the chemical-liquid injection.

An opening is formed at a front end of the protective cover 21, and the cylinder 22 is inserted into the protective layer 21 in a state of the nozzle portion 22b protruding out through the opening. A cover flange 21a having a ring-shaped recess which receives the flange 22a of the cylinder 22 formed therein, is formed at a tail end of the protective cover 21. Although the syringe is used upon being inserted into the protective cover 21 in the present embodiment, the protective cover 21 is not indispensable in the present invention, and the syringe may be loaded directly in the injection head 10a.

In FIG. 3A, another embodiment of the protective cover 21 is shown together with a syringe (the cylinder 22 and the plunger 23) to be combined therewith, is shown in an exploded perspective view. A portion protruding through the tail end of the plunger 23 in FIG. 3A is a protrusion 23a in the form of a flange which is to be engaged with the syringe drive mechanism of the injection head 10a.

The protective cover 21 shown in FIG. 3A has an upside piece 211 and a ring member 212. The upside piece 211 is attached to a portion of an outer peripheral surface of the cover flange 21a, facing an upper surface side of the injection head 10a in a state of the protective cover 21 mounted on the injection head 10a. The ring member 212 is attached to cover a portion of the outer peripheral surface of the cover flange 21a to which the upside piece 211 is not attached. The ring member 212 can be formed of a metal such as stainless steel (for example, SUS443JI).

The upside piece 211 may be attached to the protective cover 21 by an arbitrary means such as adhesive bonding (gluing), and from a viewpoint of preventing falling off the protective cover 21, structures shown in FIG. 3B and FIG. 3C are adopted in the present embodiment.

### (Fall-Off Prevention Structure 1)

A shoulder portion 211a extending toward both sides in a horizontal direction is formed on a base portion of the upside piece 211. Both end portions in a peripheral direction of the ring member 212 are bent inward, and the shoulder portion 211a of the upside piece 211 is held.

### (Fall-Off Prevention Structure 2)

The protective cover 21 and the upside piece 211 are fixed by a pin 213. Either only one of the abovementioned fall-off prevention structures may be adopted or both the abovementioned fall-off prevention structure may be adopted. When both the fall-off prevention structures are adopted, a bent portion of the ring member 212 also serves to prevent the pin 213 from coming off.

An anti-slip structure for the cylinder 22 (syringe) may be provided to an inner surface of the protective cover 21. In FIG. 3D, an anti-slip ring 214 which is an example of the anti-slip structure is shown. The anti-slip ring 214 is formed of an elastic member such as silicon rubber, and has a plurality of protrusions 214a protruding toward an inner side in a radial direction in order to make a partial contact with an outer peripheral surface of the cylinder 22. It is preferable that protrusions 214a are disposed at an equal interval in the peripheral direction. Moreover, it is possible to design appropriately the number and height of protrusions 214a not to hinder back and forth movement of the cylinder 22 into the protective cover 21 while exerting an effect as anti-slip.

A dent 21b which enables a user to put a finger easily on the flange 22a of the cylinder 22 inserted into the protective cover 21 may have been formed in the cover flange 21a of the protective cover 21. Accordingly, it becomes easy to draw out the cylinder 22 inserted into the protective cover 21.

Referring again to FIG. 1, the console 10b has an injection control unit 11, an input device 12, and a display device 13. The injection control unit Il controls an operation of the overall chemical-liquid injector such as determining injection conditions such as an injection volume and an injection rate of a chemical liquid by using at least a part of data input from the input device 12, controlling an operation of the injection head 10a so that the chemical liquid is injected according to the injection conditions determined, and controlling a display of the display device 13. It is possible to configure the injection control unit 11 by including a so-called microcomputer, and can have a CPU, a ROM, and a RAM, and an interface with other instruments. A computer program for a control of the chemical-liquid injector 10 is installed in the ROM. The CPU, by executing various functions according to the computer program, can control an operation of each section of the chemical-liquid injector 10. Moreover, to be able to carry out manual injection by user besides the automatic injection by the injection control unit 11, a hand switch is connected to the console 10b. The hand switch is provided with a start button, and by the user operating the start button, a chemical liquid is injected according to the operation.

The input device 12 is a device used for inputting data which is to be used for determining the conditions for injecting a chemical liquid by the injection control unit 11. As the input device 12, it is possible to use at least one type of known input devices such as various buttons, a keyboard, and a mouse. Data input from the input device 12 is transmitted to the injection control unit 11, and data to be displayed on the display device 13 is transmitted from the injection control unit 11. The display device 13, by being controlled by the injection control unit 11, carries out display of data necessary for determining the conditions for injecting a chemical liquid, display of an injection protocol, display of various guidance, and display of various warnings.

The display protocol is a protocol indicating as to which chemical liquid, of how much volume, and at what rate is to be injected. The injection rate may be constant or may change with time. Moreover, in a case of injecting a plurality of types of chemical liquids such as a contrast medium and a physiological saline solution, information such as, in which order the chemical liquids are to be injected, is also included in the injection protocol. As the injection protocol, it is possible to use a known arbitrary injection protocol. Moreover, regarding a procedure for setting the injection protocol, it is possible to use a known procedure, and it is also possible to make an arrangement such that the user can change arbitrarily the injection protocol that has been set. Furthermore, the injection protocol sometimes includes a maximum permissible value of injection pressure (pressure limit). In a case in which, the pressure limit has been set, during the injection operation, the injection pressure is monitored, and the operation of the injection head 10a is controlled such that the injection pressure does not exceed the pressure limit set.

As the display device 13, it may be a known display apparatus such as a liquid crystal display apparatus. Moreover, it is also possible to use a touch panel in which a touch screen is disposed as an input device on a display which is a display device, as the input device 12 and the display device 13. A part of the input device 12 may be provided separately from the console.

The chemical-liquid circuit 30 forms flow channels of a chemical liquid connecting the syringe and the patient, and can have at least one tube, at least one connecter, and at least one valve.

### [B] Configuration of Chemical-Liquid Circuit

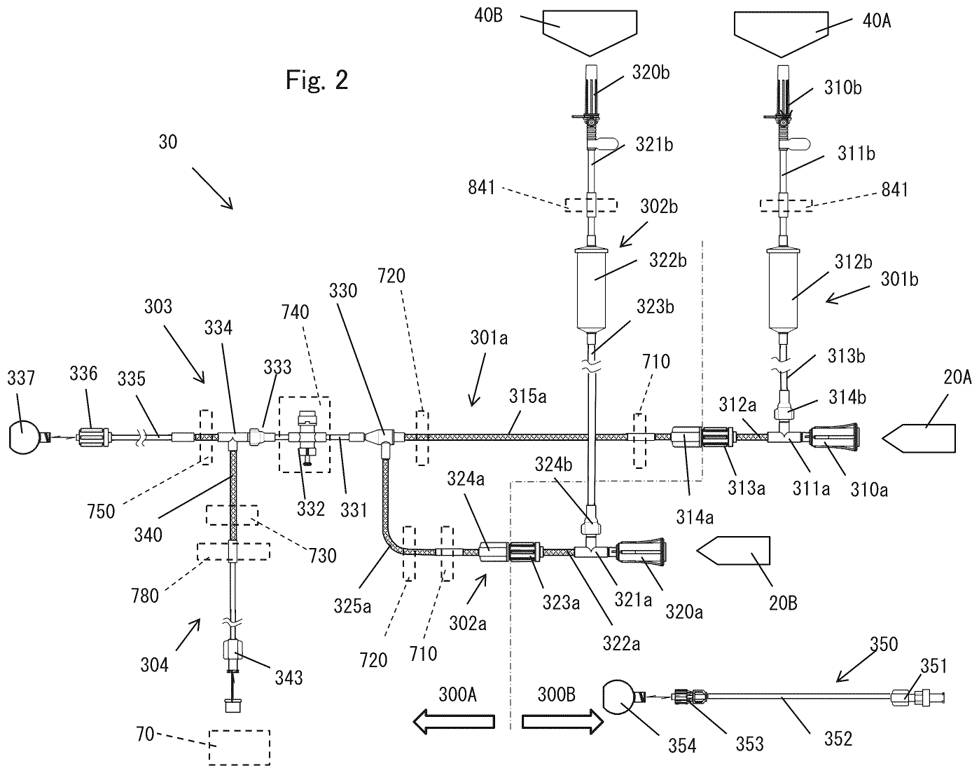

An embodiment of the chemical-liquid circuit 30 which can be used appropriately in the chemical-liquid injector 10 shown in FIG. 1, is shown in FIG. 2. In the chemical-liquid circuit 30 shown in FIG. 2, the syringes 20A and 20B are connected and are used at the time of injecting a first chemical liquid and a second chemical liquid accommodated in the respective syringes 20a and 20B into the patient. Moreover, the chemical-liquid circuit 30 is capable of connecting a first container 40A and a second container 40B accommodating the first chemical liquid and the second chemical liquid respectively, and is also capable of sucking the first chemical liquid and the second chemical liquid from the first container 40A and the second container 40B into the syringes 20A and 20B respectively. The first chemical liquid and the second chemical liquid are chemical liquids for a medical purpose, and a case in which the first chemical liquid is a contrast medium and the second chemical liquid is a physiological saline solution will be described below.

The chemical-liquid circuit 30 has a first main line 301a which is connected to the syringe 20A containing the contrast medium, a second main line 302a which is connected to the syringe 20B containing the physiological saline solution, a first sub line 301b which is connected to the first container 40a containing the contrast medium, a second sub line 302b which is connected to the second container 40B containing the physiological saline solution, a patient line 303 which located at a downstream of the first main line 301a, and a transducer line 304 which is connected to a transducer.

Here, ‘line’ signifies a flow channel through which the chemical liquid flows, and includes members (such as, various types of tubes, T-shaped tubes, various fluid connectors, various valves, and mixing devices) through which the chemical liquid flows. Moreover, in FIG. 2, each line is indicated conveniently for illustrating diagrammatically, and a relative length of each line do not represent a relative length of an actual line. Moreover, regarding the ‘tube’ which is one of the components constituting the ‘line’, the term ‘tube’ used in the following description may be formed by a single tube member, or may be formed by a tube assembly to which a plurality of tube members is connected.

The first main line 301a has, in order from an upstream sider, a syringe connector 310a, a T-shaped tube 311a, a first tube 312a, a rotating high-pressure adapter 313a, a female lure lock connector 314a, and a second tube 315a. The syringe connector 310a is rotatably connected to the T-shaped tube 311a via a rotary joint, and the syringe 20A is connected detachably. The rotating high-pressure adapter 313a and the female lure lock connector 314a are connected detachably. Accordingly, the first main line 301a is separable between the first tube 312a and the second tube 315a.

The first sub line 301b connects the first container 40A and the first main line 301a. The first sub line 301b has, in order from the first container 40A side, a spike 310b, a third tube 311b, a drip chamber 312b, a fourth tube 313b, and a unidirectional valve 314b. The spike 310b is connected to the first container 40A. The unidirectional valve 314b is installed in a direction allowing a flow of a liquid only in a direction from the first container 40A toward the first main line 301a, and is connected to the T-shaped tube 311a of the first main line 301a. The first container 40A is a container in the form of a bottle for example, and the contrast medium flowed from the first container 40A, after being dripped in the drip chamber 312b, is supplied to the first main line 301a.

As described above, by disposing the unidirectional valve 314b in the first sub line 301b, the chemical liquid is prevented from inflowing to the first sub line 301b from the first main line 301a.

The second main line 302a has, in order from the upstream side, a syringe connector 320a, a T-shaped tube 321a, a first tube 322a, a rotating high-pressure adapter 323a, a female lure lock connector 324a, and a second tube 325a. The syringe connector 320a is rotatably connected to the T-shaped tube 321 a via a rotary joint, and the syringe 20B is connected detachably. The rotating high-pressure adapter 323a and the female lure lock connector 324a are connected detachably. Accordingly, the second main line 302a is separable between the first tube 322a and the second tube 325a.

The second sub line 302b connects the second container 40B and the second main line 302a. The second sub line 302b has, in order from the second container 40B side, a spike 320b, a third tube 321b, a drip chamber 322b, a fourth tube 323b, and a unidirectional valve 324b. The spike 320b is connected to the second container 40b. The unidirectional valve 324b is installed in a direction allowing a flow of a liquid only in a direction from the second container 40B toward the second main line 302a, and is connected to the T-shaped tube 321a of the second main line 302a. The second container 40B is a container in the form of a bag for example, and the physiological saline solution flowed from the second container 40B, after being dripped in the drip chamber 322b, is supplied to the second main line 302a.

As described above, by disposing the unidirectional valve 324b in the second sub line 302b, the chemical liquid is prevented from inflowing to the second sub line 302b from the second main line 302a.

The patient line 303 has, in order from the upstream side, a mixing device 330, a fifth tube 331, a flow channel opening/closing valve 332, a unidirectional valve 333 connected via a tube to the flow channel opening/closing valve 332, a T-shaped tube 334, a sixth tube 335, and a connector 336. The mixing device 330 has two inflow ports and one outflow port, and is configured such that the liquids inflowed through the port are mixed and outflow through the outflow port. The inflow ports of the mixing device 330 are connected to the second tube 315a of the first main line 301a and the second tube 325a of the second main line 302a respectively. The outflow port of the mixing device 330 is connected to the fifth tube 331. As the mixing device 330, it is possible to use ‘SPIRAL FLOW’ (registered trademark) manufactured by Nemoto Kyorindo Co., Ltd. Moreover, it is also possible to use a T-shaped connector instead of the mixing device 330. A three-way stopcock (not shown in the diagram) may be attached to the downstream side of the sixth tube 335. A position of the three-way stopcock may be at any of the upstream and downstream of the connector 336. By the patient line 303 having the three-way stopcock at the downstream side of the sixth tube 335, it is possible to connect a hand operated syringe to the three-way stopcock according to requirement, and to inject various chemical liquids manually.

The flow channel opening/closing valve 332 is a unit configured to be capable of controlling opening and closing of flow channels. The flow channel opening/closing valve 332 will be described later in detail.

The unidirectional valve 333 is installed in a direction allowing a flow of a liquid only in a direction from the upstream toward the downstream. The connector 336 is disposed at a downstream end of the patient line 303, and the patient line 303 is connected to a patient tube such as catheter etc. that is tapped or inserted into the patient, via the connector 336. A liquid pool cap 337 may be installed at the downstream of the connector 336. The liquid pool cap 337 is a member which prevents the physiological saline solution outflowing from a downstream end of the patient line 303 by receiving the excessive physiological saline solution at the time of filling the chemical-liquid circuit 30 with the physiological saline solution for air venting of the chemical-liquid circuit 30. After the completion of air venting, the liquid pool cap 337 is removed and the connector 336 and the patient tube are connected.

The transducer line 304 is a line connected to the T-shaped tube 334 of the patient line 303 so as to branch from the patient line 303, and has, in order from the T-shaped tube 324 side, a tube assembly 340 which consists of one tube or a plurality of tubes connected in series, and a connector 343. At least a part of the tube assembly 340 has flexibility to the extent that it can be flattened by being pinched from an outer side. A transducer 70 is connected to the connector 343 for monitoring a pulse by detecting a blood pressure of the patient. A display (not shown in the diagram) which displays a pulse waveform of the patient is connected to the transducer 70.

It is possible to divide the chemical-liquid circuit 30 configured as mentioned above into a single-time use section 300A on the downstream side and a multiple-times use section 300B on the upstream side. The single-time use section 300A is a section that can be used only once, and is a so-called disposable section. The multiple-times use section 300B is a section that can be used repeatedly for a plurality of times. Specifically, the single-time use section 300A is configured to include a portion on the downstream side separated by the female lure lock connector 314a of the first main line 301a, a portion on the downstream side separated by the female lure lock connector 324a of the second main line 302a, and the patient line 303 and the transducer line 304. The multiple-times use section 300B includes a portion other than the single-time use section 300A of the chemical-liquid circuit 30, or in other words, a portion on the upstream side of the first main line 301a separated by the rotating high-pressure adapter 313a, the first sub line 301b, a portion on the upstream side of the second main line 302a separated by the rotating high-pressure adapter 323a, and the second sub line 302b.

As mentioned above, the chemical-liquid circuit 30 has a plurality of tubes, and as it will be described later in detail, there is a portion of a tube which is flattened by the flattening mechanism, and moreover, the multiple-times use section 300B is used repeatedly for a plurality of times. Therefore, for improving durability, it is preferable that at least some of these tubes are formed of blade tubes. Examples of tubes to be preferably formed by the blade tubes are cited below.

Multiple-times use section 300B:
the first tube 312a of the first main line 301a,
the first tube 322a of the second main line 302a,
   Single-time use section 300A:
the second tube 315a of the first main line 301a,
the second tube 325a of the second main line 302a,
at least the portion of the sixth tube 335 of the patient line 303 flattened by the flattening mechanism,
at least the portion of the tube assembly 340 of the transducer line 304 flattened by the flattening mechanism.

Moreover, the chemical-liquid circuit 30 can further have an auxiliary circuit 350. The auxiliary circuit 350 has a female lure lock connector 351 with a unidirectional valve, a tenth tube 352, a male lure lock connector 353 connected to the female lure lock connector 351 via the tenth tube 352, and a liquid pool cap 354 which is detachably connected to the male lure lock connector 353. The unidirectional valve of the female lure lock connector 351 allows a flow of a liquid only in a direction from the female lure lock connector 351 to the male lure lock connector 353. After the multiple-times use section 300B is connected to the syringes 20A and 20B and the chemical liquid containers 40A and 40B, air venting is carried out. In the auxiliary circuit 350, the female lure lock connector 351 is connected to each of the rotating high-pressure adapter 313a of the first main line 301a and the rotating high-pressure adapter 323a of the second main line 302a till the air venting comes to an end. The liquid pool cap 354, similarly as the liquid pool cap 337 of the aforementioned patient line 303, is a member which receives an excessive physiological saline solution at the time of air venting.

After the end of the air venting, the auxiliary circuit 350 is detached from the first main line 301a and the second main line 302a, and the female lure lock connectors 314a and 324a of the single-time use section 300A are connected to the rotating high-pressure connectors 313a and 323a respectively.

The single-time use section 300A is replaced by a new one after examination, and since the air venting of the multiple-times use section 300B is carried out even at this time, the auxiliary circuit 350 becomes necessary. However, the multiple-times use section 300B is already filled almost with the physiological saline solution from the second examination onward, a small volume of physiological saline solution is adequate for air venting. Therefore, for the auxiliary circuit 350 to be used before the second examination onward, it is possible to use a circuit in which the liquid pool cap 354 is connected directly to the lure connector 351 with the unidirectional valve.

The main configuration of the chemical-liquid circuit 30 will be described below in more detail.

### (B-a) Flow Channel Opening/Closing Valve

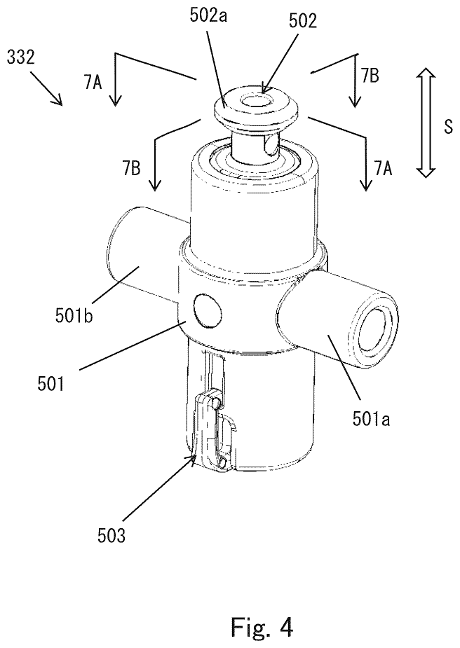

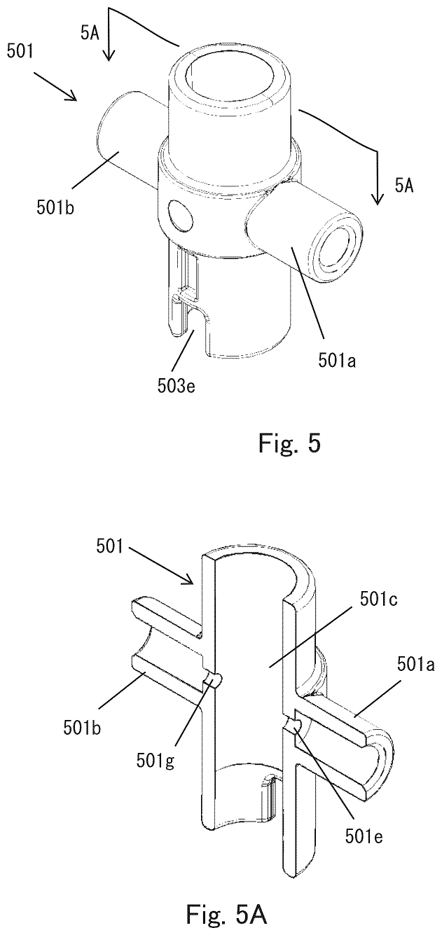

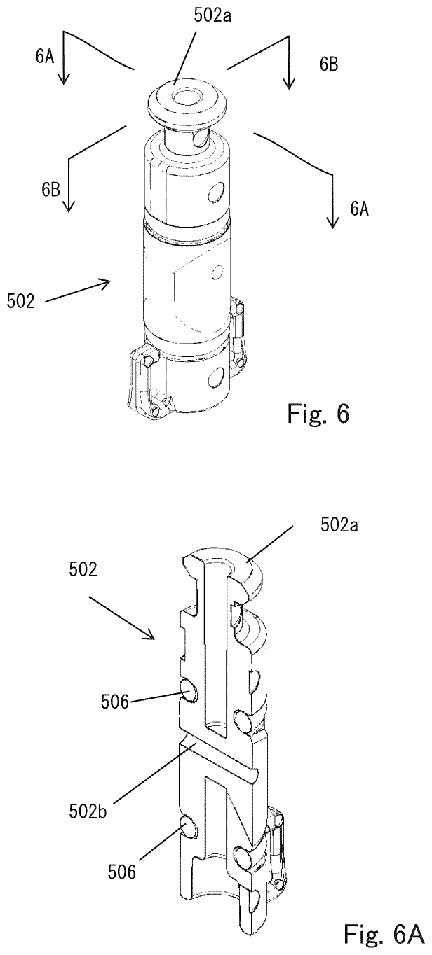

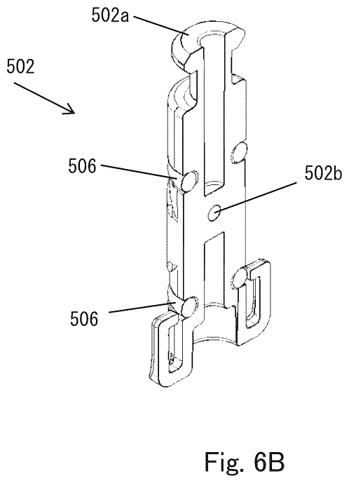

As shown in FIG. 4, the flow channel opening/closing valve 332 has a housing 501 and a piston 502 which is inserted into the housing 501 to be slidable in a direction of arrow S to be able to assume an open position and a closed position. The flow channel opening/closing valve 332 shown in FIG. 4 will be described below while referring to FIG. 5, FIG. 5A, FIG. 6, FIG. 6A, and FIG. 6B.

It is possible to mold the housing 501 by injection-molding a resin for example, and the housing 501 has a cylinder portion 501c through which the piston 502 can be slidably inserted. The cylinder portion 501 is formed by a through hole made along an axial direction of the housing 501. Moreover, the housing 501 is provided with conduit portions 501a and 501b extended from an outer wall of the housing 501 in a direction orthogonal to the axial direction of the cylinder portion 501c, adjacent to the cylinder portion 501c. One conduit portion 501a is connected to the fifth tube 331 (refer to FIG. 2) of the patient line 303. The other conduit portion 501b is connected to the unidirectional valve 333 of the patient line 303 via a tube (refer to FIG. 2). Therefore, in the embodiment illustrated in the diagram, the conduit portion 501a becomes an inflow flow channel and the conduit portion 501b becomes a discharge flow channel.

Furthermore, a communicating flow channel 501e which makes the one conduit portion 501a and the cylinder portion 501c communicate, and a communicating flow channel 501g which makes the other conduit portion 501b and the cylinder portion 501c communicate, are formed in the housing 501. The conduit portions 501a and 501b and the communicating flow channels 501e and 501g are disposed to be aligned in straight line.

The piston 502 is a columnar-shaped member molded by injection molding a resin for example, and has on one end thereof a head 502a having a flange shape spread outward in a radial direction. On an intermediate portion in a longitudinal direction of the piston 502, a flow channel 502b is formed across the piston 502 in a direction orthogonal to the longitudinal direction of the piston 502. On an outer peripheral surface of the piston 502, a sealing ring 506 such an O-ring is installed on both sides of the flow channel 502b in the longitudinal direction of the piston 502.

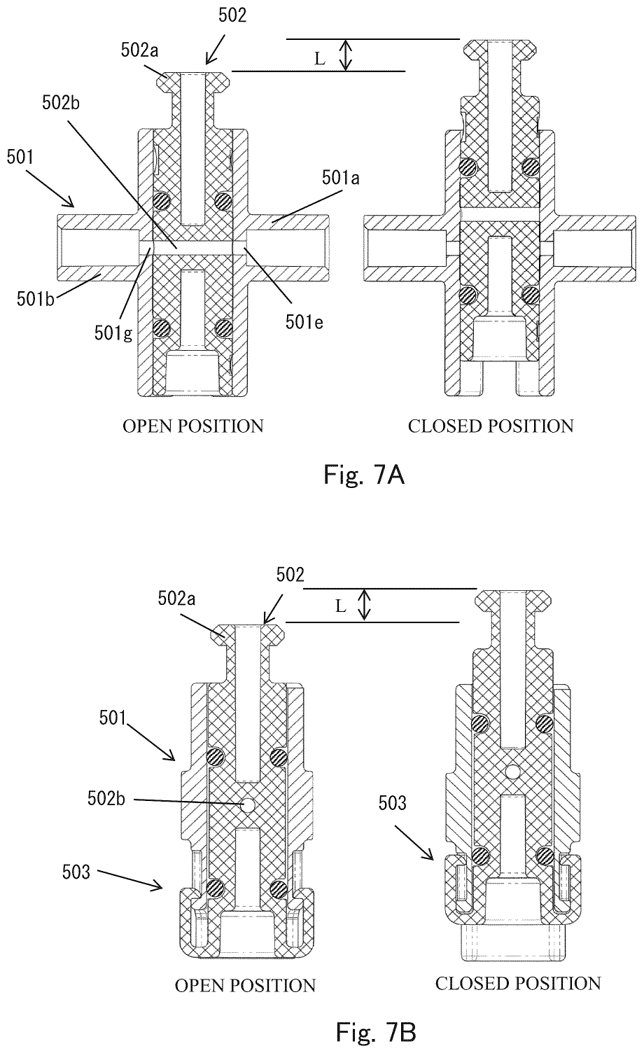

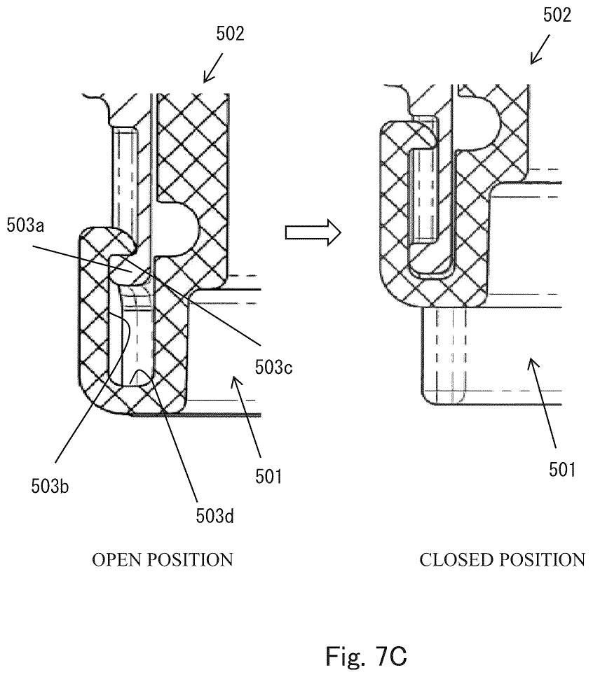

The flow channel opening/closing valve 332 further has a stopper structure 53 which limits a range of movement of the piston 502 with respect to the housing 501. The stopper structure 503, as shown in FIG. 7C, has a protrusion 503a formed on the housing 501 and a recess 503b formed on the piston 502. The recess 503b is extended in a direction of sliding of the piston 502 and receives the protrusion 503a. Moreover, a length of the recess 503b in the direction of sliding of the piston 502 is determined to have a first end portion 503c with which the protrusion 503a abuts when the piston 502 is at the open position and a second end portion 503d with which the protrusion 503a abuts when the piston 502 is at the closed position.

The protrusion 503a can be formed as a portion of the housing 501, and the recess 503b can be formed as a portion of the piston 502. Accordingly, it is possible to configure the stopper structure 503 only with the housing 501 and the piston 502 without using other components, and to configure the flow channel opening/closing valve 332 with fewer components.

In the present embodiments, the two protrusions 503a are formed on the outer peripheral surface of the housing 501 at equal angular interval in a peripheral direction. Corresponding to the two protrusions 503a, the two recesses 503b are formed on the piston 502 at equal angular interval in a peripheral direction of the piston 502. The number of protrusions 503a may be one, or may be three or more than three. The number of recesses 503b as well, may be the same as the number of protrusions.

In a case in which the protrusion 503a is formed on the outer peripheral surface of the housing 501, it is preferable to form the recess 503b, as shown in the diagram, in the form of a hook turned around the outer peripheral surface of the housing 501, to be extended from one end portion of the piston 502 (in the embodiment shown in the diagram, although the recess 503b is formed on an end portion on an opposite side of the head 502a of the piston 502, the recess 503b may be formed at an end portion of the head 502a side) beyond a peripheral wall of the housing 501.

Accordingly, it is possible to form the protrusion 503a to be received in the recess 503b. Moreover, by forming the recess 503b in the form of a hook, it is possible to attach the housing 501 and the piston 502 by a snap-fit connection without using a component other than the housing 501 and the piston 502. Moreover, it is possible to configure the flow channel opening/closing valve 332 compactly by cutting a notch 503e in the peripheral wall of the housing 501 as shown in FIG. 5, and making an arrangement such that the recess 503b is turned around the outer peripheral surface of the housing 501 upon passing through the notch 503e.

In the present embodiment, the protrusion 501a is formed on the housing 501 and the recess 503b is formed on the piston 502, but it may be reversed. In other words, the recess may be formed on the housing 501 the protrusion 501a may be formed on the piston 502. In this case, the recess can be formed on an inner peripheral surface of the housing 501 and the protrusion can be formed on the outer peripheral surface of the piston 502.

The operation of the flow channel opening/closing valve 332 will be described below by referring to FIG. 7A which is a linear cross-sectional view along a line 7A-7A of the flow channel opening/closing valve 332 shown in FIG. 4, and FIG. 7B which is a linear cross-sectional view along a line 7B-7B of the flow channel opening/closing valve 332 shown in FIG. 4.

At an open position (in the embodiment shown in the diagram, a state in which the piston 502 is slid (pushed) into the housing 501), a flow channel 502b of the piston 502 is positioned colinearly in a straight line with the communicating flow channels 501e and 502g of the housing 501, and the conduit portions 501a and 501b communicate via the flow channel 501b. Moreover, at the open position, the first end portion 503c of the recess 503b of the piston 502 abuts with the protrusion 503a of the housing 501, and an amount of movement of the piston 502 in a direction of sliding is restricted such that the piston 502 is not slid (pushed) any more into the housing 501.

As the piston 502 is drawn out from the housing 501 at the open position, the flow channel 502b moves together with the movement of the piston 502. As the piston 502 is drawn out only by a distance L from a closed position and positioned at the closed position, the communicating flow channels 501e and 501g are completely blocked by the piston 502, and a flow of a fluid between the conduit portions 501a and 501b is cut off.

Moreover, the recess 503b of the piston 502 also moves along the direction of the movement of the piston 502 together with the movement of the piston 502 from the open position to the closed position, and the protrusion 503a abuts with the second end portion 503d of the recess 503b at the closed position. Accordingly, an amount of movement of the piston 502 in the direction drawing out is restricted such that the piston 502 is not drawn out any more from the housing 501.

By configuring the stopper structure 503 which limits a range of movement of the piston 502 with respect to the housing 501 by a portion of the housing 501 and a portion of the piston 502, it is possible to configure the flow channel opening/closing valve 332 by four components which are the housing 501, the piston 502, and two sealing rings 506 at least. Moreover, the stopper structure 503 being a structure in which an engagement of the protrusion 503a and the recess 503b is used, by inserting the piston 502 into the housing 501, and then engaging the protrusion 503a and the recess 503b by snap-fitting, it is possible to assemble the flow channel opening/closing valve 332 without using an adhesive. It is possible to reduce significantly the cost of the flow channel opening/closing valve 332 of the present embodiment, as it is possible to configure by using the minimum number of components and/or to reduce the number of man-hours for assembling.

For the communicating flow channels 501e and 501g and the flow channel 502b to be cut off assuredly at the closed position, it is desirable that the inner peripheral surface of the housing 501 and the outer peripheral surface of the piston 502 make a contact at least at the closed position. For this, a packing can be added to the outer peripheral surface of the piston 502 for example. Or, the piston 502 and the housing 501 can also be formed of materials having different coefficient of elasticity. In this case, the packing is not necessary. In a case of forming the housing 501 and the piston 502 of materials having different coefficient of elasticity, the housing can be formed of a polycarbonate (PC) and the piston can be formed of a high-density polyethylene (HDPE).

It is possible to carry out the sliding (pushing) operation and drawing operation of the piston into and out of the housing 501 by using the head 502a. Specifically, in a state in which the housing 501 is fixed, it is possible to move the piston 501 between the open position and the closed position by engaging with the head 502a an engaging member which is operated to reciprocate in a direction of movement of the piston 502 by being engaged with the head 502a. For enabling the engaging member to be engaged easily with the head 502a and the piston 502 to move between the open position and the closed position, it is preferable to make an arrangement such that one end portion of the piston 502 in the direction of movement protrudes from the housing 501 in the range of movement of the piston 502 between the open position and the closed position, and to form the head 502a on the portion protruded. Or, it is also possible to make an arrangement such that both end portions of the piston 502 in the direction of movement protrude from the housing 501 in the range of movement of the piston 502 between the open position and the closed position, or to make an arrangement such that only one end portion of the piston 502 protrudes at the open position, and only the other end portion of the piston 502 protrudes at the closed position. In this case, since it is possible to move the piston 502 between the open position and the closed position by sliding (pushing) the respective pistons 502 from both sides of the piston 502, a structure such as the head 502a in which the engaging member is engaged is unnecessary. It is possible to use a sliding member in the form of a rod for sliding (pushing) the piston 502. When a pushing member having a diameter same as or smaller than a diameter of the cylinder portion 501c is used, it is possible to make a length of the piston 502 such that the piston 502 does not stick out (protrude) from the piston 502 both at the open position and the closed position.

As described heretofore, the flow channel opening/closing valve 332 of the present embodiment has the housing 501, the piston 502 which is slidably inserted into the housing 501, and the stopper structure 503 which limits the amount of sliding of the piston 502, by configuring the stopper structure 503 by the housing 501 and the piston 502, it is possible to achieve a stable operation in which there is no leakage of a chemical liquid even in an operation under high pressure.

The flow channel opening/closing valve 332 is not restricted to a valve having the abovementioned arrangement, and may have another arrangement. Instead of the flow channel opening/closing valve 332, an opening/closing unit described in International Unexamined Patent Application Publication No. 2018/181270 can be used, and a flattening mechanism described later can also be used for example.

### (B-b) Unidirectional Valve

As mentioned above, the first sub line 301b, the second sub line 302b, and the patient line 303 have the unidirectional valves 314b, 324b, and 333 respectively. Some embodiments of a unidirectional valve suitable for use under high pressure as in a chemical-liquid circuit will be described below.

### [Embodiment 1 of Unidirectional Valve]

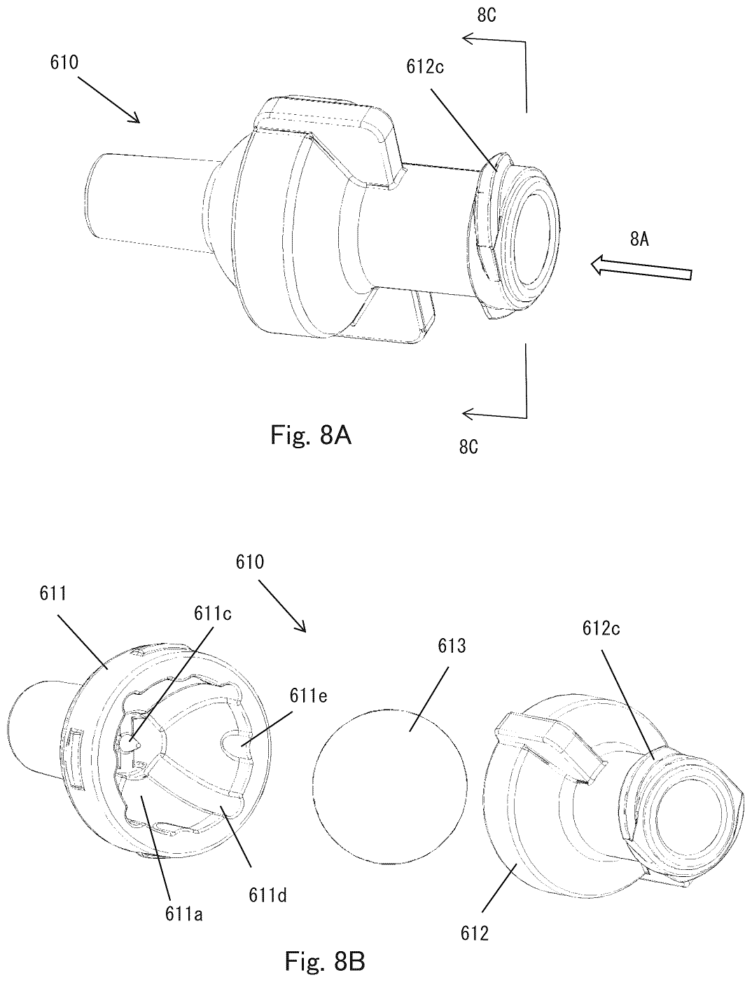

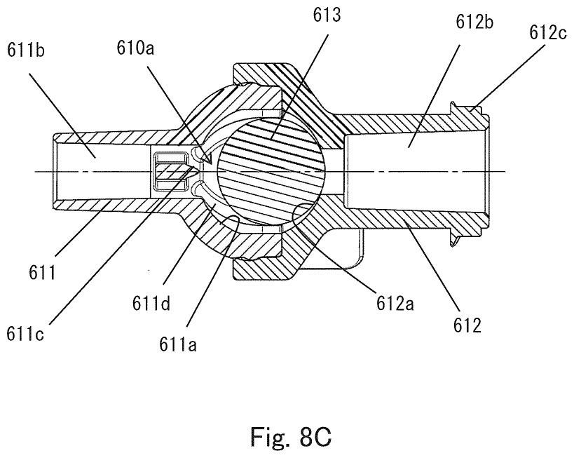

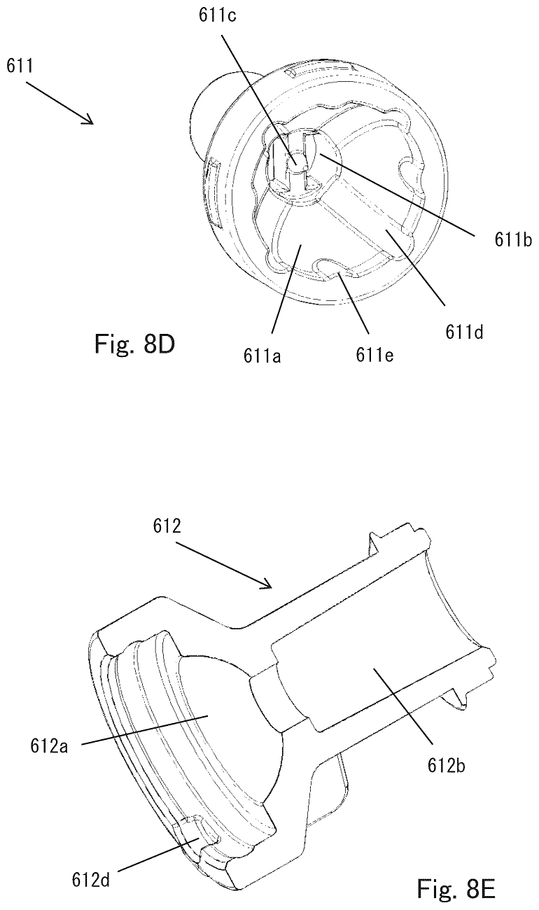

Referring to FIG. 8A, a perspective view of a unidirectional valve 610 according to an embodiment 1 is shown. Moreover, FIG. 8B shows an exploded perspective view of the unidirectional valve 610 shown in FIG. 8A, and FIG. 8C shows a cross-sectional view of the unidirectional valve 610 cut along a line 8C-8C in FIG. 8A.

The unidirectional valve 610 according to the embodiment 1 has a first case 611, a second case 612, and a valve element 613, and is configured to allow a flow of a chemical liquid only in a direction of arrow 8A. The valve element 613 is a spherical member and is movably disposed inside a valve chest 610a which is formed by joining the first case 611 and the second case 612 by fitting.

The first case 611, as shown in FIG. 8B, FIG. 8C, and Fig .8D which is a perspective view from an angle other than that of FIG. 8B, has a receiving recess 611a, a downstream-side flow channel 611b, and a valve-element position regulating protrusion 611c. The receiving recess 611a, as a whole, has a concave spherical surface, and receives the spherical-shaped valve element 613. The downstream-side flow channel 611b communicates with the receiving recess 611a, and opens at an end portion of the first case 611 on a side opposite to a joining portion with the second case 612. The valve-element position regulating protrusion 611c, by abutting by the valve element 613 that moved toward downstream side in a direction of flow of the chemical liquid (direction of the arrow 8A) inside the unidirectional valve 610, regulates a position of the valve element 613 such that the valve element 613 does not block the downstream-side flow channel 611b.

For securing satisfactorily the flow channel for the chemical liquid in the unidirectional valve 610, a groove 611d and/or a protrusion 611e may have been formed on a surface of the receiving recess 611a. It is preferable that the groove 611d is formed throughout the entire receiving recess 611a, along the direction of flow of the chemical liquid inside the unidirectional valve 610. The projection 611e, if protruding with respect to the surface of the receiving recess 611a, may have been formed throughout the entire receiving recess 611a or may have been formed only on a part of the receiving recess 611a in the direction of flow of the chemical liquid inside the unidirectional valve 610. The number of the grooves 611d and the number of the protrusions 611e may be one or more than one. The number of the grooves 611d and the number of the protrusions 611e may be same or may be different. In a case of forming the grooves 611d in plurality, it is preferable to dispose the plurality of grooves 611d at equal angular interval a peripheral direction of the unidirectional valve 610, and accordingly, there is no unevenness in the movement of the chemical liquid inside the valve chest 610a, thereby enabling to move the chemical liquid smoothly through the valve element 613 inside the valve chest 610a. Even in a case of forming the plurality of protrusions 611e, the same is applicable as in the case of forming the plurality of grooves 611d.

The second case 612, as shown in FIG. 8C and FIG. 8E which is a perspective view cut along the line 8C-8C in FIG. 8A, has a valve seat 612a and an upstream-side flow channel 612b. The valve seat 612a has a concave spherical surface. The upstream-side flow channel 612b communicates with the valve seat 612a and opens at an end portion of the second case 612 on a side opposite to joining portion with the first case 611.

In the abovementioned arrangement, when a pressure on an upstream side of the direction of flow of a liquid inside the unidirectional valve 610 becomes higher than a pressure on the downstream side due to reasons such as the inside of the valve chest 610a has a negative pressure, the valve element 613 moves inside the valve chest 610a from a second case 612 side to a first case 611 side, and accordingly, the fluid can flow inside the unidirectional valve 610. In other words, the unidirectional valve 610 opens. Whereas, when a pressure on the upstream side of the direction of flow of the liquid inside the unidirectional valve 610 becomes lower than the pressure on the downstream side due to reasons such as the inside of the valve chest 610a has a positive pressure, the valve element 613 moves inside the valve chest 610a from the first case 611 side toward the second case 612 side, and accordingly, the valve chest 610a is blocked. In other words, the unidirectional valve 610 closes.

Here, as aforementioned, the receiving recess 611a of the first case 611 has a concave spherical surface as a whole, and the valve seat 612a of the second case 612 has a concave spherical surface. Moreover, the valve element 613 is a spherical-shaped member, Therefore, the structure is such that, in an open state of the unidirectional valve 610, when a fluid flows through the valve chest 610a which is formed by the receiving recess 611a and the valve seat 612a, turbulence is not susceptible to occur at the interior of the 610a, and generation of air bubbles is suppressed. Accordingly, for instance, when a chemical-liquid container such as a chemical-liquid bottle is connected to the upstream side of the unidirectional valve 610 and an empty syringe is connected to the downstream side, and the chemical liquid is sucked in to the syringe from the chemical-liquid container, it is possible to suppress an inflow of air bubbles into the syringe.

Moreover, when the unidirectional valve 610 moves from the open state to the closed state, for the valve element 613 to move smoothly in accordance with a change in pressure of the fluid between the upstream side and the downstream side, it is preferable that the valve element 613 is formed of a material having a specific gravity smaller than a specific gravity of the fluid that flows through the unidirectional valve 610. For example, in a case of using this unidirectional valve 610 in a chemical-liquid circuit for contrast medium and physiological saline solution, the valve element 613 can be formed of polypropylene (PP). Accordingly, it is possible to prevent a reverse flow of the fluid.

A lure lock type connecting structure is formed at an upstream-side end portion of the second case 612. A lure lock type connector to be connected is installed also on a tube to be connected to the second case 612, and arrangement is such that a user can attach and detach the tube to and from the second case 612.

A method for joining the first case 611 and the second case 612 is not restricted in particular, and a method such as joining by using an adhesive, and by welding such as thermal welding and ultrasonic welding can be cited as examples thereof. In a case of joining the first case 611 and the second case 612 by an adhesive, for example, a recess which functions both as an inlet and a reservoir for the adhesive to a joining portion of the first case 611 and the second case 612 can be formed on any one of the first case 611 and the second case 612. In the embodiment 1 of the unidirectional valve 610, the portion joining the first case 611 and the second case 612 is configured such that an outer peripheral surface of the first case 611 and an inner peripheral surface of the second case 612 are joined at mutually-facing end portion side. Therefore, in the embodiment 1, a recess 612d is formed on the second case 612 as shown in FIG. 8E.

For joining the first case 611 and the second case 612, to start with, the first case 611 is fitted to the second case 612 in a state of the valve element 613 disposed between the first case 611 and the second case 612. Thereafter, an adhesive is poured into the recess 612d of the second case 612. As the adhesive is poured into the recess 612d, due to capillary action, the adhesive poured enters into the joining surfaces of the first case 611 and the second case 612. Furthermore, when the first case 611 is rotated in the peripheral direction of the unidirectional valve 610, the adhesive entered spreads in the peripheral direction and is eventually applied to joining surfaces of the first case 611 and the second case 612 throughout the periphery. By drying the adhesive in this state, the first case 611 and the second case 612 are joined. Accordingly, it is possible to join the first case 611 and the second case 612 easily and assuredly.

As described heretofore, according to the present embodiment, by joining the first case 611 and the second case 612 by fitting, it is possible to secure a wide joining area of both the first case 611 and the second case 612, and by forming the valve element 613 by a spherical-shaped member, it is possible to achieve a stable operation without occurrence of a leakage of the chemical liquid even in an operation under high pressure.

In the embodiment 1, to enable to install the tube detachably on the second case 612, the lure-lock type connecting structure is formed on the upstream-side end portion of the second case 612. However, this lure-lock type connecting structure is not indispensable, and the second case 612 and the tube may have been attached (glued).

### [Embodiment 2 of Unidirectional Valve]

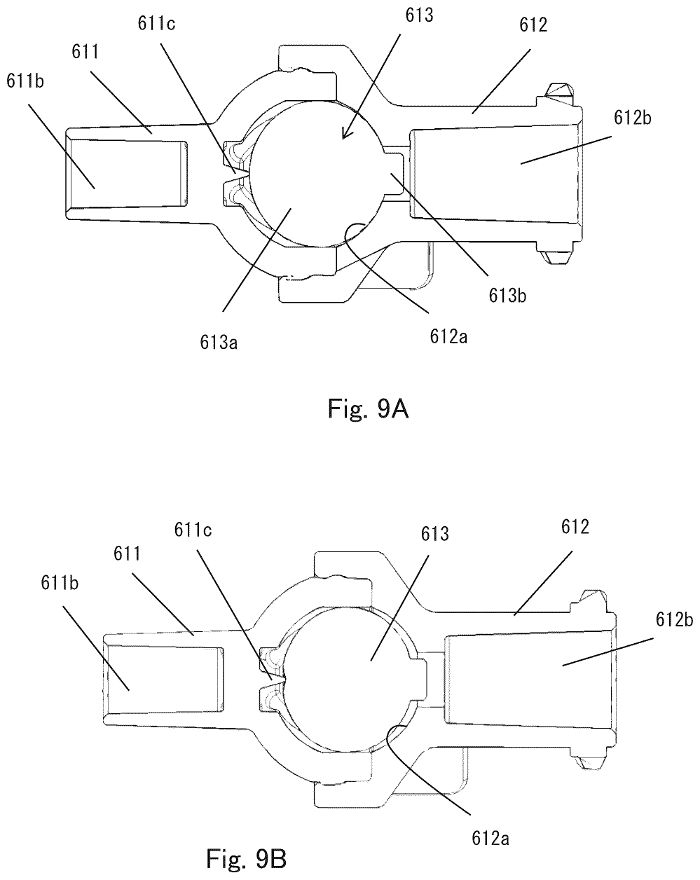

In FIG. 9A and FIG. 9B, cross-sectional views along a direction of flow of a fluid of an embodiment 2 are shown. FIG. 9A and FIG. 9B show a closed state and an open state respectively of the unidirectional valve. The basic configuration of the embodiment 2 is same as that of the embodiment 1, and has the first case 611, the second case 612, and the valve element 613. However, in the embodiment 2, the size and material of the valve element differ from those in the embodiment 1. Description of the arrangement which may be same as that in the embodiment 1 is omitted, and mainly the valve element 613 will be described below.

In the embodiment 2, the valve element 613 has a body 613a which is a spherical portion, and a protrusion 613b which is a portion protruding from the body 613a. The protrusion 613b is positioned inside a communicating hole which makes communicate a valve chest and the upstream-side flow channel 612b, and has a size and shape which allow a flow of fluid between the valve chest and the upper-side flow channel 612b.

Such protrusion 613b can be used for holding the valve element 613 at a fixed position at the time of joining the first case 611 and the second case 612 during the assembling of the unidirectional valve 610. For instance, the valve element 613 is disposed with respect to the second case 612 to be directed such that the protrusion 613b is positioned inside a second communicating hole when the valve element 613 is made to abut with the valve seat 612a, and in this state, a valve-element fixing jig in the form of a long and thin rod (not shown in the diagram) is inserted into the upstream-side flow channel 612b from a side of the second case 612 opposite to the valve seat 612a. A front end of the valve-element fixing jig has a structure that enables to fix the protrusion 613b, and the valve element 613 is fixed to the valve-element fixing jig inserted. Accordingly, since the valve element 613 is held) at a fixed position with respect to the second case 612, by joining the second case 612 and the first case 611 in this state, it is possible to assemble the valve element (unidirectional valve) 610 more easily. After assembling the valve element (unidirectional valve) 610, the valve element fixing jig is removed from the valve element 613.

The body 613a which is a spherical portion of the valve element 613 has a diameter larger as compared to that in the embodiment 1, and a valve-element occupancy which is a proportion of a volume of the body 613a in a volume of the valve chest is higher as compared to that in the embodiment 1. The valve-element occupancy can be expressed by Vb/Vc×100 (%) when the volume of the valve chest is let to be Vc, and the volume of the body 613b of the valve element 613 is let to be Vb. By making the valve-element occupancy high, stagnation of air in the valve chest is suppressed, and it is possible to improve venting of air by the flow of the fluid inside the unidirectional valve 610. For suppressing the stagnation of air in the valve chest effectively, it is preferable that the valve-element occupancy is 80% or higher, and valve-element occupancy of 85% or higher is more preferable, and 90% or higher is even more preferable. However, when the valve-element occupancy is excessively high, the fluid cannot flow easily, and therefore, it is preferable that the valve-element occupancy is not higher than 95%.

When the valve-element occupancy is let to be the maximum in a practical range, it is possible to make a size of the valve element 613 such that the valve element 613 makes a contact with the valve-element position regulating protrusion 611c at the downstream side and with the valve seat 612a at the upstream side. In this case, the movement of the valve element 613 in the valve chest being difficult, the valve element 613 is formed of an elastic member such as rubber, and an arrangement is made such that the unidirectional valve 610 opens by the valve element 613 being deformed elastically with a rise in pressure acting on the valve element 613 at the upstream side of the valve element 613. The elastic deformation of the valve element 613, to be more precise, is compression, and as shown in FIG. 9B, the valve element 613 is compressed toward the downstream side, thereby moving away from the valve seat 612a, and the valve element 613 opens thereby. In contrast, as the pressure acting of the upstream side of the valve element 613 returns to normal, the shape of valve element 613 is restored, and the valve element 613 makes a contact with the valve seat 612a. The unidirectional valve 610 is closed by the valve element 613 making a contact with the valve seat 612a.

Silicon rubber can be used as a material of the valve element 613. For the elastic deformation of the valve element 613 to occur with the change in pressure, it is preferable that the degree of hardness of the valve element 613 is 70 degrees or less, and the degree of hardness of 50 degrees or less is more preferable. On the other hand, as an excessive elastic deformation of the valve element 613 occurs with the change in pressure, when the valve element 613 has compressed with the rise in pressure on the upstream side, the valve element 613 makes a contact with an inner surface of the valve chest throughout the peripheral direction of the unidirectional valve 610 thereby blocking the valve chest, and leading to a possibility of a defective opening/closing operation. For preventing such defective opening/closing operation, it is preferable that the degree of hardness is 30 degrees or higher, and the degree of hardness of 40 degrees or higher is more preferable.

The embodiment 2 of the unidirectional valve 610 has heretofore been described. In the abovementioned description, the protrusion 613b of the valve element 613 is not indispensable, and it may be only a spherical portion similarly as in the embodiment 1. Conversely, in the embodiment 1, the valve element 613 may have the protrusion 613b similarly as in the embodiment 2.

### [Embodiment 3 of Unidirectional Valve]

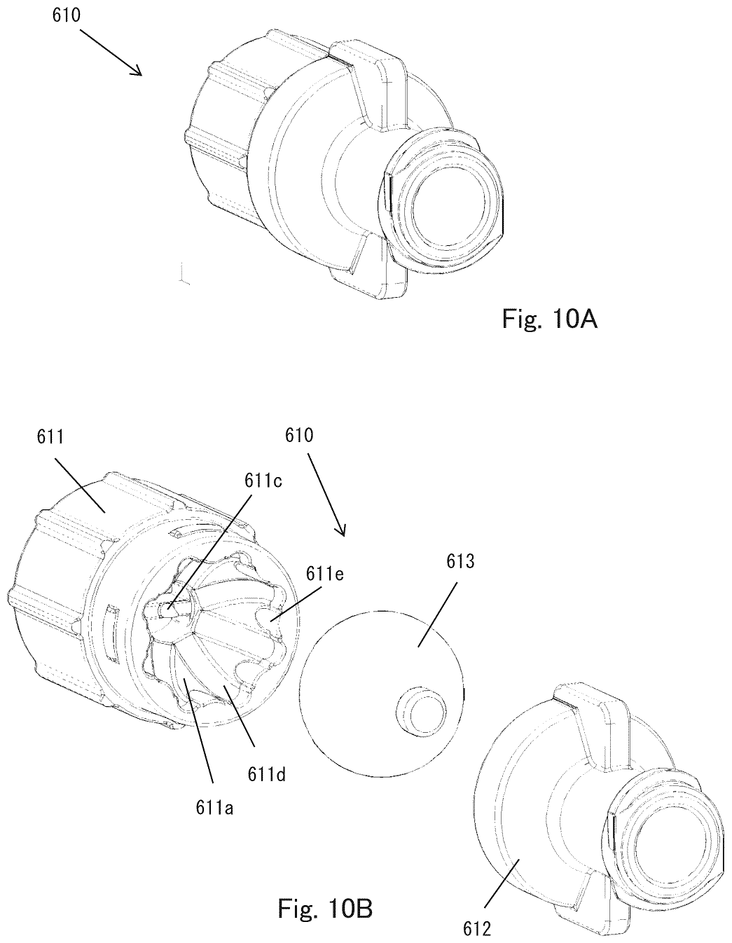

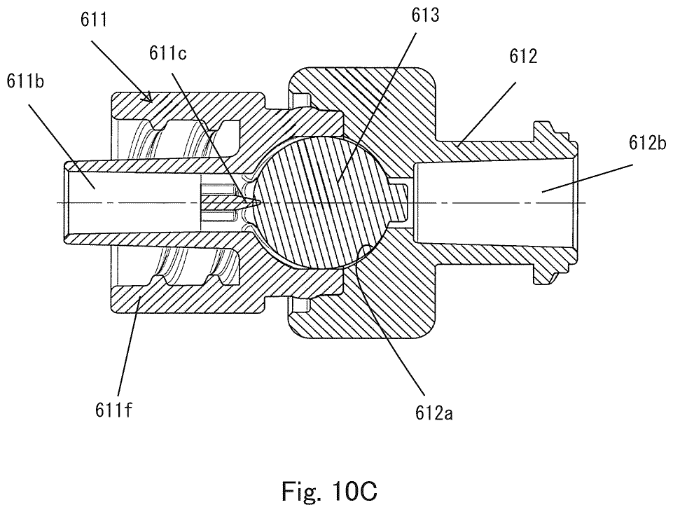

In FIG. 10A, a perspective view of an embodiment 3 of a unidirectional valve is shown, and in FIG. 10B, an exploded perspective view thereof is shown. Moreover, in FIG. 10C, a cross-sectional view along a direction of flow of a fluid in the unidirectional valve of the embodiment 3 is shown. The basic configuration of the embodiment 3 is same as that of the embodiment 2, and the unidirectional valve has the first case 611, the second case 612, and the valve element 613. However, in the embodiment 3, a structure of the first case 611 differs from that in the embodiment 2. Description of the arrangement which may be same as that in the embodiment 2 is omitted, and mainly the first case 611 will be described below.

The first case 611, as shown in FIG. 10C, is configured to have a structure which functions as a lure lock connector. A lure lock connector to be connected to this connector is installed also on a tube which is connected to this first case 61, and an arrangement is made such that the user can attach and detach the tube to and from the first case 611. As described in the embodiment 1, since the second case 612 is also configured such that, the tube can be attached and detached, according to the embodiment 3, the user can remove the unidirectional valve 610 freely from the tube. Such first case 611 having a structure which functions as a connector is also applicable to the embodiment 1.

### [Embodiment 4 of Unidirectional Valve]

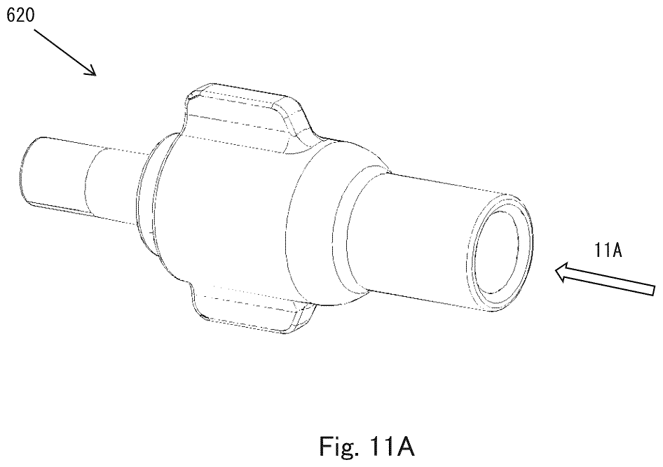

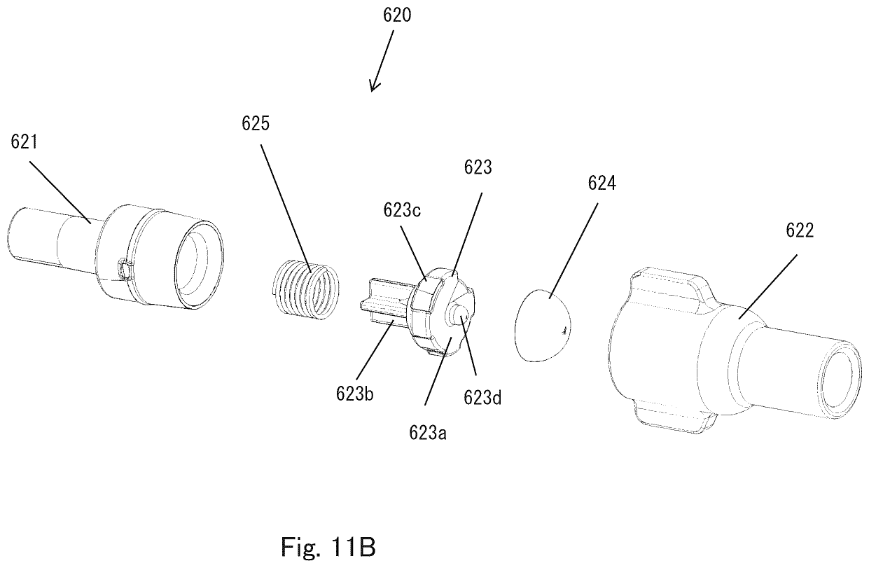

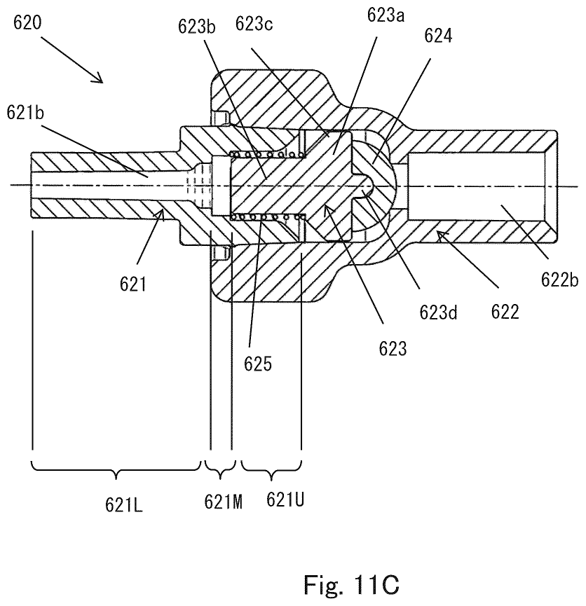

In FIG. 11A, a perspective view of an embodiment 4 of the unidirectional valve is shown, and in FIG. 11B, an exploded perspective view thereof is shown. Moreover, in FIG. 11C, a cross-sectional view along a direction of flow of fluid in the unidirectional valve of the embodiment 4 is shown. A unidirectional valve 620 of the embodiment 4, similar to the embodiments 1 to 3, has a first case 621, a second case 622, and a valve element 623, and allows a flow of a chemical liquid only in a direction of arrow 11A, but a shape and structure of each differ from those in the embodiments 1 to 3, and the unidirectional valve 620 in the embodiment further has a gasket 624 and a bias applying spring 625.

The first case 621 and the second case 622 have a downstream-side flow channel 621b and an upstream-side flow channel 622b respectively, and form a valve chest by being joined mutually. A method for joining and a structure of the joint of the first case 621 and the second case 622 may be similar to those of the embodiment 1. The valve element 623, the gasket 624, and the bias applying spring 625 are disposed inside the valve chest.

The valve element 623 has a valve body 623a, a leg 623b, and a plurality of first projections 623c and second projections 623d, and is positioned inside a valve chest to be movable in an axial direction of the unidirectional valve 620. The valve body 623a is a portion of the valve clement 623, of which a downstream-side end portion is formed to be tapered. The leg 623b is a portion of the unidirectional valve 620 extended from a downstream-side end surface of the valve body 623a in an axial direction of the unidirectional valve 620. The first projection 623c is formed on a side surface of the valve body 623a, and prevents movement of the valve body 623 a in a radial direction of the unidirectional valve 620. The second projection 623d is formed on an upstream-side end surface of the valve body 623a for holding the gasket 624.

The gasket 624 is a hemispherical member and is disposed such that, a hemispherical surface is directed toward the upstream side in order to be able to block an opening on a valve chest side of the upstream-side flow channel 622b by the hemispherical surface. A recess is formed on a downstream-side end surface of the gasket 624, and the gasket 624 is held by the valve body 623 a by the second projection 623d of the valve element 623 fitting in this recess. A hemispherical apex of the gasket 624 may have a protrusion similar to the protrusion 613b of the valve element 613 described in the embodiment 2 (refer to FIG. 9A).

For the bias applying spring 625, an arbitrary means can be used provided that it is capable of applying bias to the valve element 623 toward the upstream side of the unidirectional valve 620. A coil spring is used in the embodiment 4, and the leg 623b is positioned on an inner side of the coil spring. A spring coefficient of the bias applying spring 625 is set in accordance with a predetermined pressure that has to carry out the opening/closing operation of the unidirectional valve 620. By setting appropriately the spring coefficient of the bias applying spring 625, it is possible to use as the unidirectional valve 620 which opens at the time of suction of a chemical liquid from a chemical-liquid container to a syringe, or, as a unidirectional valve which opens at the time of injecting the chemical liquid from the syringe.

The downstream-side flow channel 621b of the first case 621 is formed to be stepped to have a first portion 621U having a diameter decreasing gradually from the upstream side toward the downstream side, a second portion 621M, and a third portion 621L. The first portion 621U has a diameter through which the bias applying spring 625 can be inserted, but the valve body 623a cannot be inserted. The second portion 621M has a diameter through which the leg 623b can be inserted, but the bias applying spring 625 cannot be inserted. The third portion 621L has a diameter through which the leg 623b cannot be inserted. Therefore, a step between the first portion 621U and the second portion 621M functions as a stopper stopping the bias applying spring, and a step between the second portion 621M and the third portion 621L functions as a stopper stopping the leg 623b. However, for reducing a pressure loss inside the unidirectional valve 620, it is preferable to design dimensions of components such as the leg 623b and the bias applying spring 625 such that a dimension in a radial direction of these steps becomes minimum. Moreover, making a spatial volume inside the unidirectional valve 620 as small as possible is preferable for suppressing the stagnation of air inside the unidirectional valve 620, and to improve the venting of air.

Based on the abovementioned configuration, when the pressure acting on the upstream side of the unidirectional valve 620 is lower than or equal to a predetermined pressure, the bias is applied to the valve element (valve body) 623a toward the upstream side by the bias applying spring 625, and an opening on the valve chest side of the upstream-side flow channel 622b is blocked by the gasket 624. In other words, the unidirectional valve 620 is closed. When the pressure acting on the upstream side of the unidirectional valve 620 becomes higher than a predetermined pressure, or when the pressure in the valve chest becomes a negative pressure, the valve element 623 moves toward the downstream side resisting the bias applied by the bias applying spring 625. Accordingly, the gasket 624 moves away from an opening of the upstream-side flow channel 622b on the valve chest side, and the unidirectional valve 620 opens.

### (B-c) Additional Unit

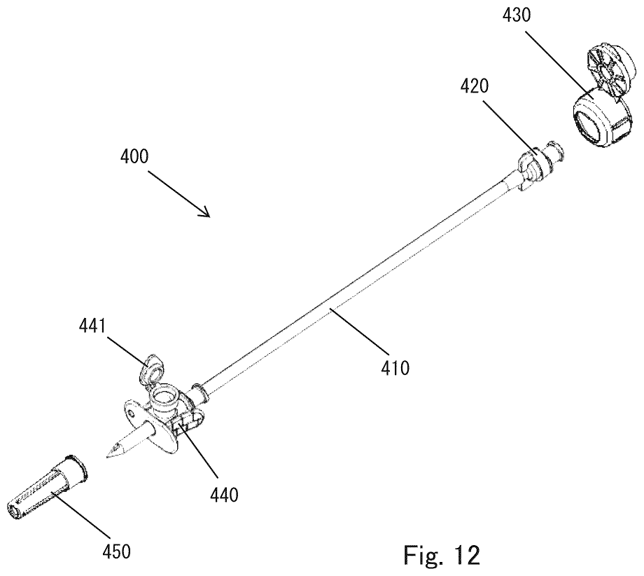

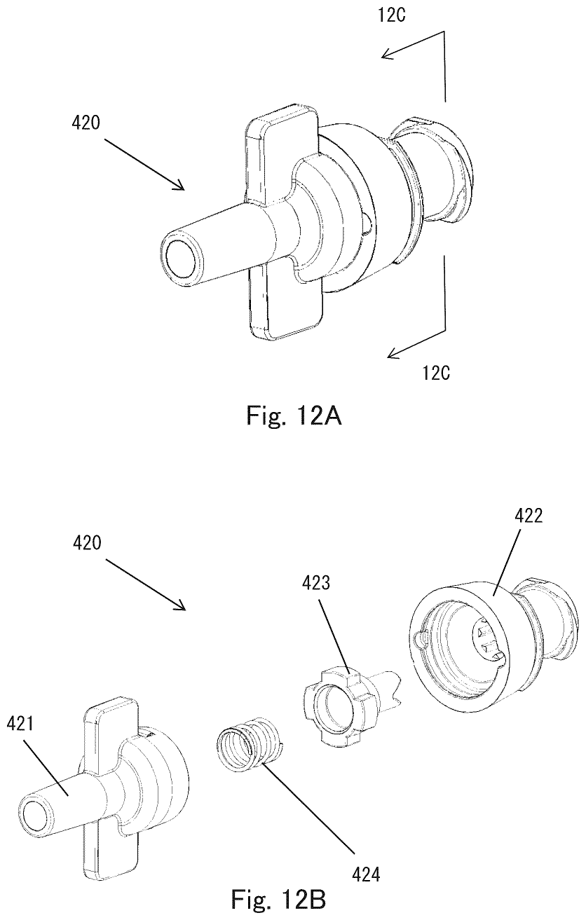

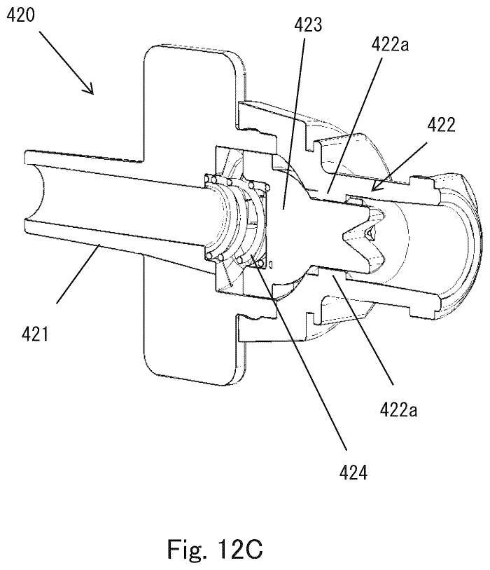

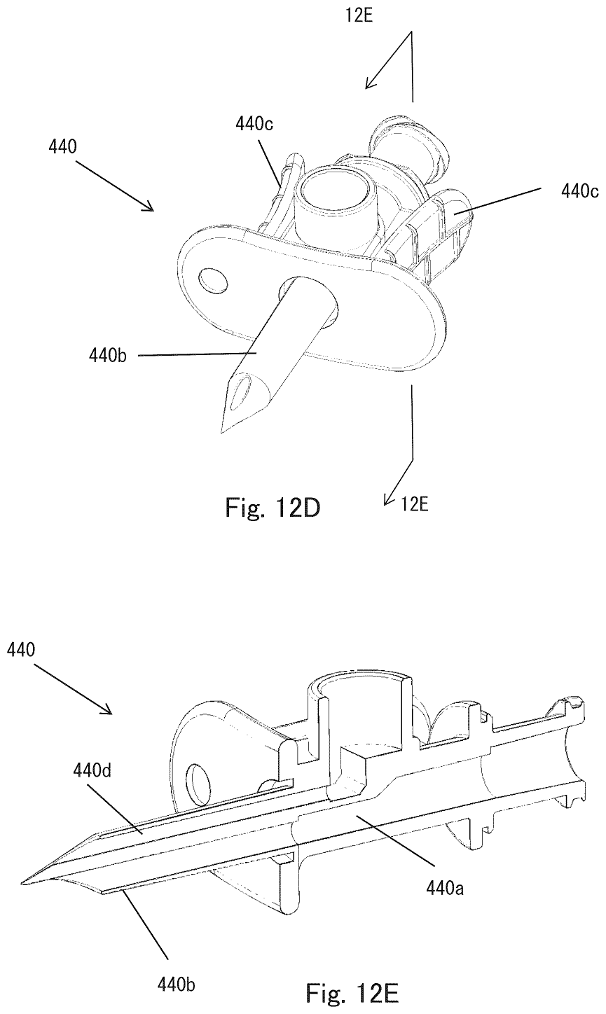

The chemical-liquid circuit 30 may further include an additional unit. A suction tube unit which is used for sucking a chemical liquid from chemical-liquid containers (40A and 40B) to syringes (20A and 20B) can be cited as an example of the additional unit. An embodiment of the suction tube unit will be described below by referring to FIG. 12 which is a perspective view thereof, and FIG. 12 A to FIG. 12E which are perspective views of components forming the suction tube unit.

A suction tube unit 400 has a tube body 410, a suction valve 420 which is connected to one end of the tube body 410, an opening/closing dust cap 430 with a lid which is detachably installed at an end of an opening of the suction valve 420, a spike 440 which is connected to the other end of the tube body 410, and a spike cap 450.

The suction valve 420 has a first case 421. a second case 422, a valve element 423, and a coil spring 424. Each of the first case 421 and the second case 422 has a flow channel, and by the first case 421 and the second case 422 being joined, a valve chest which communicates with these flow channels is formed between the flow channels of the first case 421 and the second case 422.

The valve element 423 and the coil spring 424 are disposed inside the valve chest. Inside the valve chest, a bias directed toward the flow channel of the second case 422 is applied to the valve element 423 by the coil spring 424. The valve element 423, due to the bias applied by the coil spring 424 blocks an opening of a flow channel to the valve chest of the second case 422, but when a force pushing the valve element 423 toward the first case 421 acts on the valve element 423, the valve element 423 moves to the first case 421 side resisting the bias applied by the coil spring 424, and accordingly, an opening on the valve chest side of the flow channel of the second case 422 opens. A front-end portion (end portion on the second case 422 side) of the valve element 423 is formed to have a shape such that, when a front end of a nozzle portion of the syringe has abutted, it does not block an opening of the nozzle portion, and there is a fluid communication between the interior of the syringe and the valve chest.

The flow channel of the second case 422 has a diameter that enables to insert the nozzle portion of the syringe. A plurality of ribs 422a is formed on an inner surface of the flow channel of the second case 422 at equal interval in a peripheral direction of the flow channel. These ribs 422a function as a stopper of the syringe and prevent the syringe from entering excessively deep into the suction valve 420. Furthermore, the ribs 422a function as a guide at the time of operation of the valve element 423, and accordingly, it enables to operate the valve element 423 stably.

The spike 440 has an infusing flow channel 440a for the chemical liquid which is a portion to be connected to the chemical-liquid container, extended throughout the longitudinal direction. A front-end portion of the spike 440 is formed as a sharp-pointed piercing portion 440b, and by piercing the piercing portion 440b through a plug member of the chemical-liquid container, the chemical-liquid container and the infusing flow channel are fluid-communicated. In order to enable the user to hold the spike 440 firmly during the piercing operation, it is preferable to provide integrally a pair of grippers 440c to a base of the piercing portion 440b. Anti-slip treatment by roughening may have been carried out on the gripper 440c. Moreover, an air vent 440d besides the infusing flow channel 440a is formed on the piercing portion 440b. A filter 441 (not shown in FIG. 12D and FIG. 12E) is installed at a tail end of the air vent 440d.

Here, a lure-lock type connecting structure is formed at an end portion of the second case 422. On the other hand, a lure-lock type connecting structure to be engaged with the connecting structure of the second case 422 is formed on the opening/closing dust cap 430. By these connecting structures, the opening/closing dust cap 430 is detachably connected to the end portion of the second case 422. Moreover, the spike cap 450 is detachably mounted on the piercing portion 440b of the spike 440. When the suction tube unit 400 is not in use, the opening/closing dust cap 430 and the spike cap 450 remain to be in a state of being mounted on the suction valve 420 and the spike 440 respectively, and accordingly, foreign matter is prevented from entering and mixing into the suction tube unit 400.

Next, a procedure for use of the abovementioned suction tube unit 400 will be described below. To start with, a lid of the opening/closing dust cap 430 is opened, and the spike cap 450 is removed. Next, after piercing the spike 440 through the plug member of the chemical liquid container, the nozzle portion of the syringe is inserted into the flow channel of the suction valve 420. The syringe to be used is an empty syringe without the chemical liquid filled therein, and a syringe in which a plunger is at the most forward position. By inserting the nozzle portion of the syringe into the suction valve 420, the valve element 423 is slid (pushed) down, and accordingly, the suction valve 420 opens. After connecting the chemical-liquid container and the syringe via the suction tube unit 400 in such manner, the chemical liquid is sucked into the syringe from the chemical-liquid container by retracting the plunger from the syringe. After sucking the chemical liquid, the nozzle portion of the syringe is pulled out from the suction valve 420, as well as the spike 440 is pulled out from the plug member of the chemical-liquid container. By the nozzle portion of the syringe being pulled out from the suction valve 420, the valve clement 423 is returned to the original position by the bias applied by the coil spring 424, and the suction vale 420 is closed.

### (B-d) Syringe Connector

The structure of the syringe connectors 310a and 320a connecting the syringe and the chemical-liquid circuit is not restricted in particular, provided that there is no leakage of the chemical liquid while injecting the chemical-liquid and during the suction operation, and it is preferable to have a function which prevents loosening of the syringe and the connector.

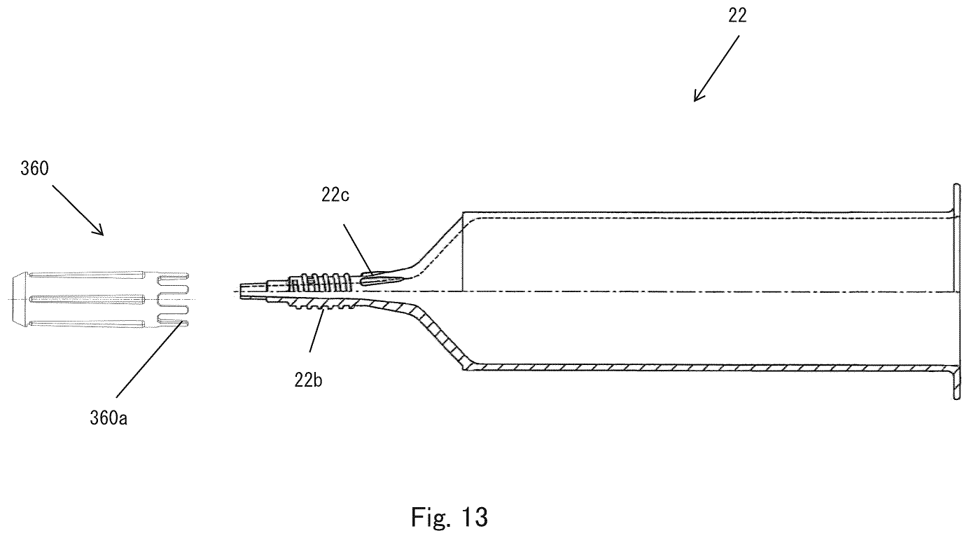

In FIG. 13, a side view of the syringe connector having the loosening prevention function and the syringe corresponding to the syringe connector are shown. In FIG. 13, a lower half of the syringe is shown in a cross-sectional view. A syringe connector 360 shown in FIG. 13 is a member in the form of a cap through which a nozzle portion of a syringe 22 is to be inserted, and a thread groove with which a screw thread formed on the nozzle portion of the syringe 22 is engaged is formed on an inner surface of the syringe connector 360, and it is possible to connect the syringe connector 360 and the syringe by screwing the syringe connector 360 in the nozzle portion of the syringe 22.

A connecting member (not shown in FIG. 13) such as a tube or a T-shaped tube is connected via a rotary joint to an end portion of the syringe connector 360, on a side opposite to a side through which the nozzle portion of the cylinder 22 is inserted. A plurality of connector protrusions 360a is formed on an end portion of the syringe connector 360, on the side through which the nozzle portion of the cylinder 22 is inserted. The connector protrusions 360a are disposed at equal interval in a peripheral direction of the syringe connector 360, and is extended in a longitudinal direction of the syringe connector 360 to further extend the syringe connector 360.

Corresponding to the connector protrusions 360a, a plurality of syringe protrusions 22c protruding from an outer surface is formed also on the nozzle portion of the syringe 22. The syringe protrusions 22c are formed at positions to have a shape and dimensions such that, the syringe protrusions 22c are positioned between the connector protrusions 360a when the syringe 22 and the syringe connector 360 are connected.

While connecting the syringe 22 and the syringe connector 360, as the syringe connector 360 is screwed in the nozzle portion of the syringe 22, the connector protrusions 360a hit the syringe protrusions 22c, and the syringe connector 360 is patiented to resistance by the syringe protrusions 22c. Here, the syringe connector 360 is configured to have a material and dimensions such that when the syringe connector 360 is rotated further, the connector protrusions 360a are deformed elastically, and can cross over the syringe protrusions 22c. Accordingly, the user is able to apply even larger force by the syringe connector 360 and to screw the syringe connector 360 in the nozzle portion of the syringe 22 by rotating further.

As the syringe connector 360 is screwed till the end and is fully connected to the syringe 22, the connector protrusions 360a are positioned between the syringe protrusions 22c. In this state, for rotating the syringe 22 and the syringe connector 360 relatively, it is necessary to apply force by which the connector protrusions 360a are deformed elastically and can cross over the syringe protrusions 22c, and this in turn, works as the function of preventing loosening of the syringe 22.

By the connector protrusions 360a crossing over the syringe protrusions 22c, the user feels a clicking sensation. By feeling the clicking sensation, the user is capable of knowing sensuously that the syringe connector 360 is screwed in the nozzle portion of the syringe 22. The magnitude of clicking sensation can be set arbitrarily by designing appropriately the material of the syringe connector 360, the dimensions of the connector protrusions 360a, and the dimensions of the syringe protrusions 22c.

### [C] Configuration of Injection Head

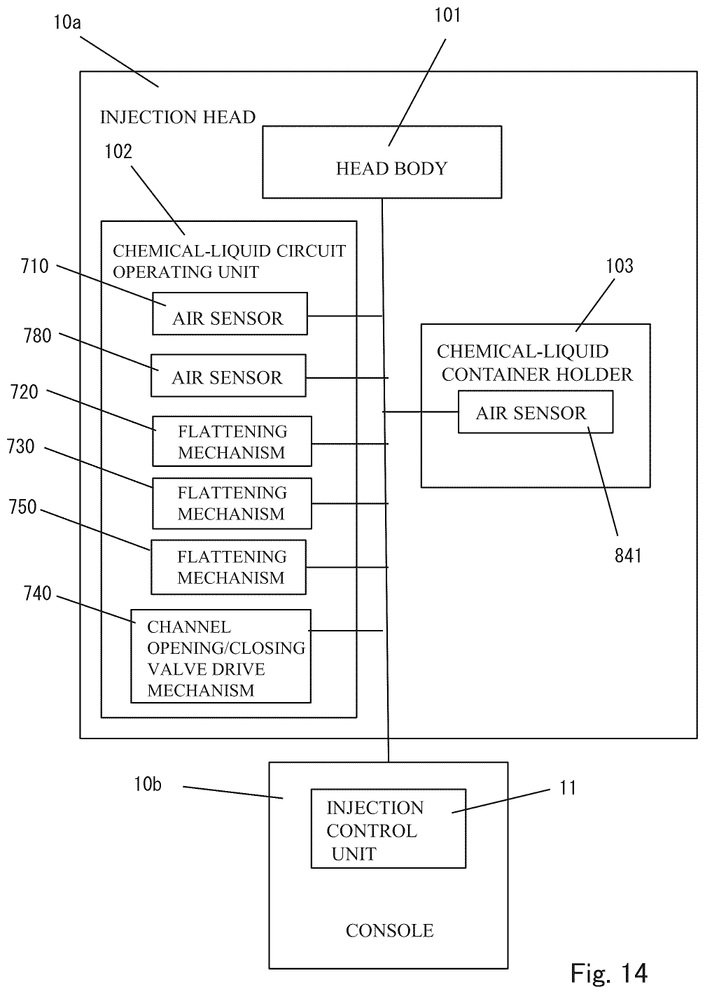

Next, the injection head 10a shown in FIG. 1 will be described below by referring to diagrams such as FIG. 14. The injection head 10a has a head body 101 in which a syringe is loaded, a chemical-liquid circuit operating unit 102 which is disposed on a front side (side on which the syringe is loaded) of the head body 101, and a chemical-liquid container holder 103 which holds the chemical-liquid containers 40A and 40B (refer to FIG. 1). The head body 101 is a portion at which there is a possibility of the chemical-liquid circuit operating unit 102 and the chemical-liquid container holder 103 coming in contact with the chemical liquid. Therefore, it is preferable that an exterior material forming these casings is formed of a resin having a superior chemical resistance such as PETCARBO (registered trademark) and Iupilon (registered trademark).

### (C-a) Head Body

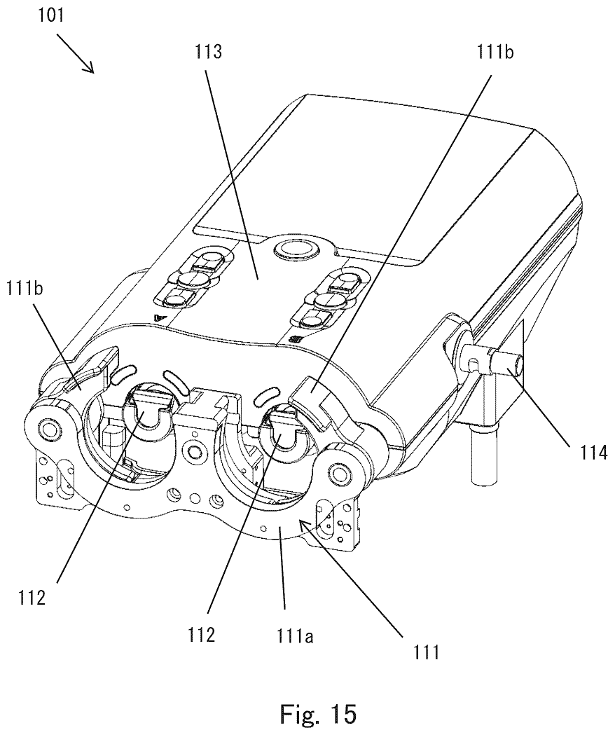

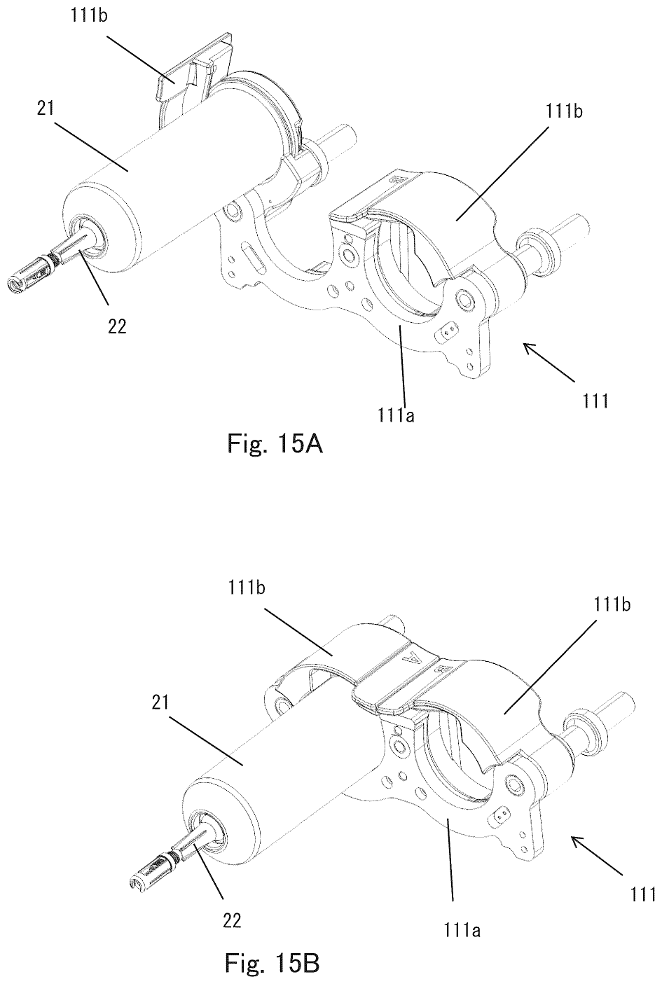

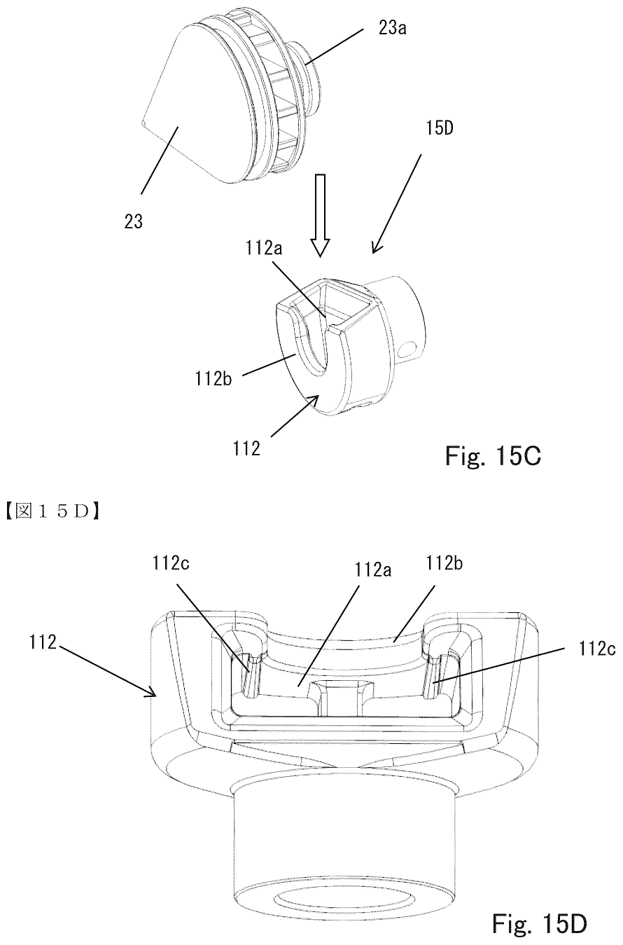

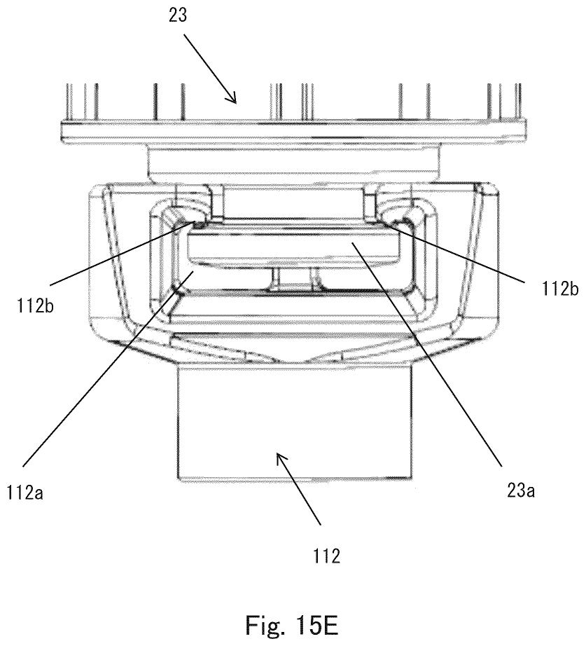

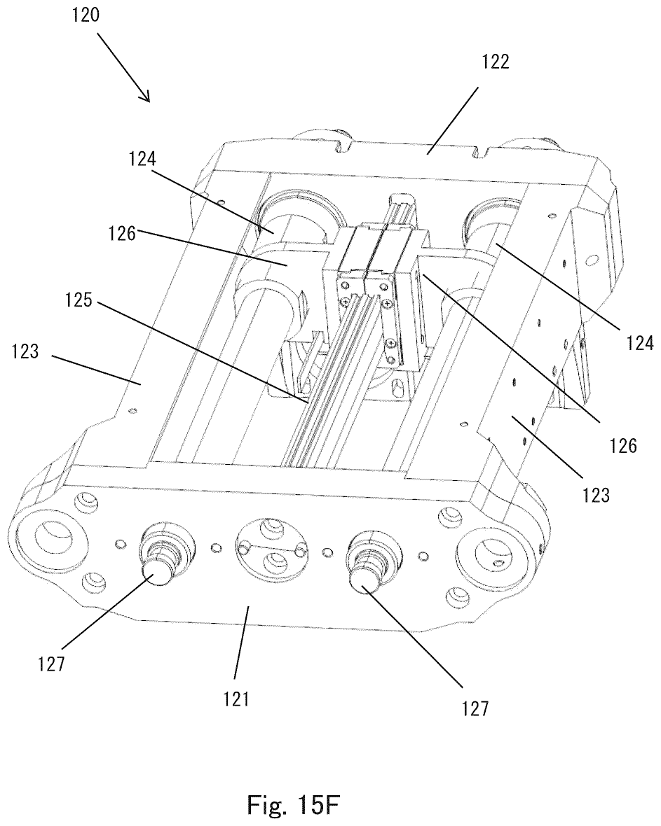

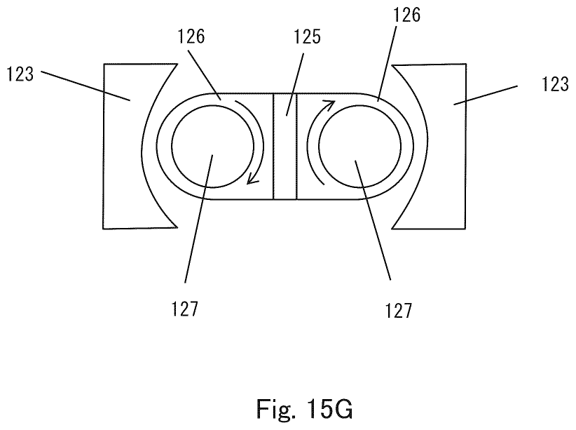

A main function of the head body 101 is loading a syringe and operating the syringe loaded. For this, the head body 101, as shown in FIG. 15, has a clamper 111 which detachably fixes two syringes 20A and 20B (refer to FIG. 1), a presser 112, and an operating section 113.

The clamper 11 can have a first holding structure 111a and two second holding structures 111b which hold the syringe in cooperation with the first holding structure 111a. The first holding structure 111a has two recesses for receiving a part in a peripheral direction of a tail end portion of two cylinders (a cover flange 21a of the protective cover 21 in a case in which the syringe is mounted with the protective cover 21 as in the embodiment shown in FIG. 3 etc.). The second holding structure 111b is disposed corresponding to each recess of the first holding structure 111a, and is configured to have a recess which is capable of receiving at least remaining part of the flange portion received by each recess.

The second holding structure 111b is movably supported between an open position and a closed position with respect to the first holding structure 111a, and at the closed position, holds the tail end portion of the syringe in cooperation with the first holding structure 111a to be immovable in the longitudinal direction of the syringe.

Here, the meaning of the term ‘to be immovable’ includes not only that the intended structure does not move at all, but also that the intended structure moves within a range of clearance developed due to a design dimensional tolerance etc.

The second holding structure 111b, as shown in FIG. 15A, is pivotably supported by the first holding structure 111a at an intermediate portion in a peripheral direction thereof. By turning the second holding structure 11b up to the closed position in a state of the syringe (and the protective cover 21) loaded on the second holding structure 111b, the syringe (and the protective cover 21) are held as shown in FIG. 15B. By making an arrangement such that the intermediate portion in the peripheral direction of the second holding structure 111b is supported by the first holding structure 111a in such manner, the compact head body 101 in which spreading in a horizontal direction when the second holding structure 111b is moved from the closed position to the open position is suppressed, is achieved. This enables loading and unloading of the syringe in a limited space.

The presser 112 is made to be movable back and forth by a drive source such as a motor, and forms a part of the syringe drive mechanism. The presser 112, at a front-end portion thereof, has an engaging portion which is to be engaged with the plunger (or piston) of the syringe. By the engaging portion being engaged with the plunger (or piston), and by moving the presser 112 back and forth in a state of the syringe being held by the clamper 11, the plunger (or piston) moves back and forth with respect to the syringe. Accordingly, it is possible to inject the chemical liquid from the syringe and to suck the chemical liquid into the syringe.

As shown in FIG. 15C to FIG. 15E, the presser 112 has a receiving recess 112a which is open upward to receive the protrusion 23a of the plunger 23 of the syringe and a hook 112b which is extended inward to be engaged between a body of the plunger 23 and the protrusion 23a. When the presser 112 moves forward and pushes the plunger 23 into the cylinder (refer to FIG. 3), a front surface of the hook 112b acts to push the plunger 23 by a surface contact. When the presser 112 retracts and draws the plunger 23 out from the cylinder 22, a rear surface of the hook 112b acts on the protrusion 23a of the plunger 23. Particularly in the embodiment shown in the diagram, for the rear surface of the hook 112b to make a point contact or a line contact with the protrusion 23a of the plunger 23, one or a plurality of protrusions 112c is formed on the rear surface of the hook 112b.

Accordingly, it is possible to suppress rattling in a frontward and rearward direction of the projection 23a inside the receiving recess 112a while reducing a frictional force which is generated between the plunger 23 and the rear surface of the hook 112b at the time of pushing and drawing the plunger 23 in and out of the receiving recess 112a. As a result, it is possible to carry out attaching and detaching of the plunger 23 smoothly to and from the presser 112, and to make the movement of the presser 112 on the plunger 23 effective more reliably.

The number and shape of the protrusions 112c provided on the rear surface of the hook 112b may be arbitrary. For instance, from a viewpoint of carrying out the operation of attaching and detaching the plunger 23 smoothly to and from the presser 112, it is preferable to let the protrusion 112c have a shape of which, a longitudinal direction is along a direction of attaching and detaching the plunger 23. Moreover, from a viewpoint of making the movement of the presser 112 on the plunger 23 effective more assuredly, it is preferable to dispose the plurality of protrusions 112c symmetrically.

As the syringe drive mechanism, an arbitrary mechanism which is capable of moving the presser in the frontward and rearward direction can be used. An embodiment of the syringe drive mechanism is shown in FIG. 15F.

A syringe drive mechanism 120 shown in FIG. 15F is a mechanism in which a ball screw mechanism is used, and has a front frame 121, a rear frame 122, a pair of left and right side frames 123, a pair of left and right ball screw mechanisms 124, a linear guide 125, a pair of left and right sliders 126, and a pair of left and right rams 127 each of which moves in the frontward and rearward direction by the operation of each ball screw mechanism 124. The linear guide 125 is a member extended in a direction of movement of the ram 127, and is disposed between the pair of side frames 123. Each ball screw mechanism 124 is disposed between the linear guide 125 and the side frame 123 to be positioned on both left and right side of the linear guide 125. Each slider 126 is disposed on both left and right sides of the linear guide 125, and is fixed to a nut of the ball screw mechanism 124, as well as, is movably supported by the linear guide 125 to be movable in the frontward and rearward direction. Each ram 127 is fixed to each slider 126, and is movably supported to be movable in the frontward and rearward direction together with the slider 126. The presser 112 (refer to FIG. 15 etc.) is fixed to a front-end portion of the ram 127. A motor is coupled to each ball screw mechanism 124, and as a ball screw of the ball screw mechanism 124 is rotated by the motor, the ram 127 moves in the frontward and rearward direction only by a distance corresponding to the amount of rotation.

According to the syringe drive mechanism 120 of the present embodiment, by supporting the pair of sliders 126 by the common linear guide 125, a configuration of the syringe drive mechanism 120 is simplified, and it is possible to configure the syringe drive mechanism compactly. Moreover, in the present embodiment, as shown in FIG. 15G, the side frames 123 are configured to have a recess which allows the slider 126 and the ram 127 to escape. Accordingly, it is possible to make small a dimension in a horizontal direction (leftward rightward direction) of the syringe drive mechanism 120, and more compact syringe drive mechanism 120 is achieved.

Moreover, in the syringe drive mechanism 120 in which the ball screw mechanism 124 is used, since a force in a direction of rotation by the rotation of the ball screw acts on the slider 126, the linear guide 125 is required to have a mechanical strength which movably supports the slider 126 so that the slider 126 does not rotate. In the present embodiment, since the pair of sliders 126 supports one linear guide 125 positioned between the sliders 126, in a case in which the ball screw mechanism 124 is operated such that both the sliders 126 move in the same direction, a force which cancels the rotative force of both the sliders 126 acts on the linear guide 125. Accordingly, the mechanical strength required for the linear guide 125 may be a mechanical strength equivalent to that for supporting one slider 125, and consequently, it is possible to use a compact and light-weight linear guide 125. This also contributes to making the syringe drive mechanism 120 compact.

The operating section 113 has a plurality of buttons such as a forward-movement button and a backward-movement button for operating the presser 112, and the user can operate the presser 112 as desired, besides operating according to the conditions set in the injection control unit 11 (refer to FIG. 1).

Moreover, the head body 101 can have a support shaft 114 which is extended in a direction orthogonal to the longitudinal direction of the syringe that is loaded. The head body 101 can be pivotably supported with the support shaft 114 as a center, by a swivel arm (not shown in the diagram) extended from a ceiling or a stand (not shown in the diagram) via the support shaft 114. By supporting the head body 101 in a state of the support shaft 114 directed in a substantially horizontal direction, it is possible to pivotably support the head body 101 between a posture in which the front end of the syringe is directed toward the ceiling (upward posture) and a posture in which the front end of the syringe is directed toward a floor (downward posture).

### (C-b) Chemical-Liquid Circuit Operating Unit

The chemical-liquid circuit operating unit 102, with the single-time use section 300A (refer to FIG. 2) of the chemical-liquid circuit 30 detachably installed therein, has a plurality of mechanisms which control the flow channels of the single-time use section 300A. These mechanisms are to be driven electrically, and in order that no chemical liquid comes in contact with these mechanisms, these mechanisms are accommodated in a casing, excluding a portion which is necessary for drawing around the chemical-liquid circuit 30.

The chemical-liquid circuit operating unit 102 may have been fixed to the head body 101. By fixing the chemical-liquid circuit operating unit 102 to the head body 101, it is possible to dispose the chemical-liquid circuit 30 in an orderly manner without the tubes in the chemical-liquid circuit 30 being bent.

Air censors 710 and 780, flattening mechanisms 720, 730, and 750, and a flow channel opening/closing valve drive mechanism 740 can be cited as the mechanisms provided to the chemical-liquid circuit operating unit 102. Operation of these mechanisms is controlled by the injection control unit 11 of the console 10b (refer to FIG. 1). Moreover, positions of these mechanism are shown in FIG. 2.

### (C-b1) Air Sensors

Air sensors detect air inside the flow channels. Referring to FIG. 2, the two air sensors 710 detect the existence of air inside second tubes 315a and 325b of the first and second main lines 301a and 302a respectively in the chemical-liquid circuit 30. The air sensor 780 detects the existence of air inside the eighth tube 340 of transducer line 304 of the chemical-liquid circuit 30. As the air sensors 710 and 780, known arbitrary sensor can be used, provided that the sensors are capable of detecting air inside the tube. An ultrasonic sensor having a transmitter and a receiver disposed face-to-face with the tube in between can be cited as an example of the air sensor.

In a case of using the ultrasonic sensor, the transmitter and the receiver are disposed face-to-face with a tube which is a portion of the flow channel, in between the transmitter and the receiver. Moreover, for detecting air more assuredly, it is essential that the tube is pinched by the air sensor making a close contact therewith. Therefore, it is preferable to configure in combination with a silicon tube such as, replacing the portion of the flow channel pinched by the air sensor by a silicon tube, or covering the outer side of the portion of the flow channel with a silicon tube.

The air sensors 710 and 780 can be provided with a light-emitting module. In this case, the air sensors 710 and 780 are arranged such that the light-emitting module is lit when the air is detected the and the light-emitting module goes out when the air is not detected. Accordingly, the user can check visually with ease that the air has been detected.

### (C-b2) Flattening Mechanism

The flattening mechanisms 720, 730, and 750 controls opening and closing of flow channels by being operated such that the tube is flattened or released to be opened. The flattening mechanisms 720 and 730 can be disposed near the air sensors 710 and 780. The flattening mechanisms 720 and 730 may have been disposed on an upstream side or on a downstream side of the air sensors 710 and 780, provided that the flattening mechanisms 720 and 730 are near the air sensor 710 and 780. It is preferable that in the first main line 301a and the second main line 302a, the flattening mechanisms 720 and 730 are disposed on a patient side (downstream side) of the air sensors 710 and 780 in order to carry out a closing operation which effectively hinders the air reaching the patient by the control of the flattening mechanisms 720 and 730 when air has been detected. Moreover, it is preferable that in the transducer line 304, the air sensor 780 is disposed on the downstream side of the flattening mechanism 730 in order to prevent an unnecessary detection of air sucked into the transducer line 304. The flattening mechanisms 720, 730, and 750 can have for instance, a base on which the tube is placed and a pushing member which is slidably supported by the base. It is possible to close the flow channel by flattening the tube by the base and the pushing member by moving the pushing member toward the base in a state of the tube disposed on the base.

### (C-b3) Flow Channel Opening/Closing Valve Drive Mechanism

The flow channel opening/closing valve drive mechanism 740 is a mechanism for opening and closing the flow channel in the flow channel opening/closing valve 332 by having the flow channel opening/closing valve 332 installed therein (refer to FIG. 4), and by driving the flow channel opening/closing valve 332.

The configuration of the flow channel opening/closing valve drive mechanism 740 is not restricted in particular, provided that it is capable of controlling opening and closing of a flow channel, and the flow channel opening/closing valve drive mechanism 740 can have a holder which detachably holds the flow channel opening/closing valve 332, the hook which is an engaging portion for engaging with the piston 502 of the flow channel opening/closing valve 332 (refer to FIG. 4), and a mechanism which moves the hook (hook moving mechanism). At the time of opening the flow channel opening/closing valve 332, in order that there is no reverse flow of the chemical liquid, it is preferable that the flow channel opening/closing valve drive mechanism 740 is controlled to open the flow channel opening/closing valve 332 after a predetermined time has elapsed after the start of injection so that a pressure is applied such that the pressure on the upstream side of the flow channel opening/closing valve 332 becomes higher than the pressure on the downstream side of the flow channel opening/closing valve 332. The flow channel opening/closing valve drive mechanism 740 can further be provided with a sensor which detects whether the flow channel opening/closing valve 332 has been installed on the flow channel opening/closing valve drive mechanism 740. Such sensor is not restricted in particular and an arbitrary sensor which is capable of detecting that the flow channel opening/closing valve 332 has been installed can be used.

In the chemical-liquid circuit 30 shown in FIG. 2, the flattening mechanism 730 is disposed in the transducer line 304, but instead of this, the flow channel opening/closing valve 332 may be disposed in the transducer line 304, and the flattening mechanism 730 may be replaced by the flow channel opening/closing valve 332.

### (C-b4) Illumination of Flow Channel Opening/Closing Valve

The chemical-liquid circuit operating unit 102 can have a lighting module which illuminates the flow channel opening/closing valve 332 installed in the flow channel opening/closing valve drive mechanism 740. Accordingly, the flow channel opening/closing valve 332 installed in the chemical-liquid circuit operating unit 102 becomes easily visible. The lighting module includes a light source, and a method of illuminating by the lighting module may be arbitrary. As the light source, it is possible to use an arbitrary light source such as a light emitting diode. It is preferable to make an arrangement such that the lighting module illuminates the flow channel opening/closing valve only when the flow channel opening/closing valve is installed in the flow channel opening/closing valve drive mechanism. Accordingly, the user can check visually with ease that the flow channel opening/closing valve has been installed in the flow channel opening/closing valve drive mechanism.

A flow channel opening/closing valve detection sensor provided with a light-emitting module can be used replacing the lighting module. The flow channel opening/closing valve detection sensor is configured to detect the flow channel opening/cloving valve 332 which is held in a state of being operable by the flow channel opening/closing valve drive mechanism 740, and the light-emitting module is lit when the flow channel opening/closing valve 332 has been detected. An arbitrary sensor such as an optical sensor or a contact sensor can be used as the flow channel opening/closing valve detection sensor.

In a case of using the air sensor 710 and 780 with the light-emitting module and the flow channel opening/closing valve detection sensor with the light-emitting module combinedly, colors of light emitted by the light-emitting modules of the two sensors may be different such as, color of light emitted by the light emitting module of the air sensors 710 and 780 is red and the color of light emitted by the light-emitting module of the flow channel opening/closing valve detection sensor is green.

Moreover, as the tube is removed from the air sensors 710 and 780, detection result same as that when air has been detected is obtained. Therefore, it is possible to use the detection result of both sensors for detecting removal of the single-time use section 300A of the chemical-liquid circuit 30. After the detection is completed, the single-time use section 300A is detached from the chemical-liquid circuit operating unit 102 in preparation for the subsequent examination. As the single-time use section 300A is detached, since the air sensors 710 and 780 change from a state of no detection of air to detection of air, and the flow channel opening/closing valve detection sensor changes from a state of detection to no detection, and accordingly, it is possible to detect the detaching of the single-time use section 300A. When the detaching of the single-time use section 300A is detected, a message indicating that the single-time use section 300A has been detached is displayed on a display device of the console. Moreover, since the new single-time use section 300A, for a sterilization process at the time of manufacturing, is let to be in a state of the flow channel opening/closing valve 332 open, as a preparation for attaching the new single-time use section 300A to the chemical-liquid circuit operating unit 102 at the time of the subsequent examination, the hook of the flow channel opening/closing valve drive mechanism 740 is operated such that the flow channel opening/closing valve 332 in the open state can be installed.

### (C-c) Chemical Liquid Container Holder

The chemical-liquid container holder 103 is a holder for detachably holding the chemical-liquid containers 40A and 40B, and can be attached in a suspended manner to the injection head 10a or the chemical-liquid circuit operating unit 102. The chemical-liquid container holder 103 may have an air sensor 841 which detects air inside the first sub line 301b and the second sub line 302b as shown in FIG. 2. An arbitrary sensor such as an ultrasonic air sensor can be used as the air sensor 841.

### [D] Operation of Chemical-Liquid Injector

Next, an operation of the abovementioned chemical-liquid injector will be described below by referring mainly to the operation of the flow channel opening/closing valve 332 and the flattening mechanisms 720, 730, and 750. These operations are controlled by the injection control unit 11. In the following description, a case in which the first main line 301a and the first sub line 301b are lines for contrast medium (A), and the second main line 302a and the second sub line 302b are for physiological saline solution (B) will be described. Moreover, for simplifying the description, contrast medium will be mentioned as ‘chemical liquid A’ and physiological saline solution will be mentioned as ‘chemical liquid B’. Moreover, in the following description, ‘the flattening mechanism opens’ signifies that the flattening mechanism is driven such that that a flow channel opens between the upstream side and the downstream side thereof. Similarly, ‘flattening mechanism closes’ signifies that the flattening mechanism is driven such that the flow channel is closed between the upstream side and the downstream side thereof. Moreover, for the transducer line 304, in a case in which the flattening mechanism 730 is replaced by the flow channel opening/closing valve drive mechanism 740, an operation of the flattening mechanism 730 in the following description can be interpreted as an operation of the flow channel opening/closing valve drive mechanism 740 that has been replaced.

### (D-a) Self Check

In a self-check to be carried out after the power supply is put ON, an opening and closing operation for verifying whether each of the flattening mechanisms 720, 730, and 750, and the flow channel opening/closing valve 332 operate normally is carried out for a multiple number of times, and all are to be opened finally. In this state, it is possible to carry out connection of the multiple-times use section 300B, connection of the single-time use section 300A, and attaching the single-time use section 300A to the chemical-liquid circuit operating unit 102.

### (D-b) While Operation is Stopped

While the operation is stopped after the completion of various operations, each of the flattening mechanisms 720, 730, and 750 is kept in an open state, and the flow channel opening/closing valve 332 is kept in a closed state. However, at the time of set-up of the multiple-times use section 300B, each of the flattening mechanisms 720, 730, and 750, and the flow channel opening/closing valve 332 is left open, and at the time of set-up of the single-time use section 300A (after installing the tube), the flow channel opening/closing valve 332, the flattening mechanism 730 of the transducer line 304, and the flattening mechanism 750 of the sixth tube 335 are kept in an open state, but an arrangement may be made such that the flattening mechanism 720 on the A side and the B side is closed.

### (D-c) Forward-Moving Operation on A Side and B Side

When the syringe drive mechanism on the A side moves forward at the time of injecting the chemical liquid A for instance, the flattening mechanism 720 on the B side and the flattening mechanism 730 of the transducer line 304 are closed, and the flow channel opening/closing valve 332 opens. When the syringe drive mechanism on the B side moves forward at the time of flushing operation by the chemical-liquid B for instance, the flattening mechanism on the A-side and the flattening mechanism 730 of the transducer line 304 close, and the flow channel opening/closing valve 332 opens. At the time of moving forward the syringe drive mechanism on the A side and the syringe drive mechanism on the B side simultaneously such as while injecting the chemical liquid A and the chemical liquid B simultaneously, the flow channel opening/closing valve 332 opens, and the flattening mechanism 730 of the transducer line 304 closes.

### (D-d) Retracting Operation on A Side and B Side

When the syringe drive mechanism on the A side retracts at the time of sucking the chemical liquid from the first container 40A to the syringe 20A, the flattening mechanism 720 on the A side closes. When the syringe drive mechanism on the B-side retracts at the time of sucking chemical liquid from the second container 40B to the syringe 20B, the flattening mechanism 720 on the B side closes. At the time of making retract both the syringe drive mechanism on the A side and the B side simultaneously, the flattening mechanism 720 on both of the A side and the B side close.

### (D-e) At the Time of Standby

At the time of standby of injection of the chemical liquid A, the flattening mechanism on the B side closes.

### (D-f) At the Time of Priming of Transducer

At the time of priming of transducer, the flattening mechanism 720 on the A side and the flattening mechanism 750 for the patient line 303 close, and the flow channel opening/closing valve 332 opens.

### (D-g) At the Time of Detection of Air

At the time of detection of air, the flow channel opening/closing valve 332 and the flattening mechanism 750 for the patient line 303 close. However, the flattening mechanism 750 for the patient line may be closed when the air is detected during standby state. Regarding the transducer line 304, the operation of the flattening mechanism 730 may be arbitrary, and in a case in which the air sensor 780 is disposed on the downstream side of the flattening mechanism 730, for preventing the sucking of air from the transducer line 304 to the patient line 303, it is preferable that the flattening mechanism 730 closes.

Here, in the chemical-liquid circuit 30 of the present embodiment, the patient line 303 has the mixing device 330. The mixing device 330 is provided with a chamber for mixing the chemical liquid A and the chemical liquid B. This chamber enables to trap for a time, the air flowed downstream from the air sensor 710 of the A side and the B side. Consequently, the risk of the air inflowing to the patient is reduced. The risk of the air inflowing to the patient is reduced by making long a length of the tube from the air sensor 710 to the flow channel opening/closing valve 332. However, in a case in which the length of the tube is restricted due to limitations on layout of the chemical-liquid circuit 30, by disposing a chamber which is capable of trapping air in a space from the air sensor 710 up to the flow channel opening/closing valve 332 as in the present embodiment, an effect similar to that in a case of using a long tube can be anticipated.

### (D-h) Miscellaneous

Regarding the timing for opening and closing the flattening mechanism 730 of the transducer line 304, it is preferable to that it closes according to injection timing, in a pressure range in which the transducer is protected. In a case in which there is a residual pressure after injecting, in order to avoid an effect of the residual pressure on the transducer, it may open after a predetermined time after the injection (in a case of a manual injection by a hand switch, after the start button of the hand switch is released).

Moreover, after various operations are completed, at the time of detaching the single-time use section 300a, the flattening mechanism 720 on the A side and the B side, the flow channel opening/closing valve 332, the flattening mechanism 730 of the transducer line 304, and the flattening mechanism 750 of the sixth tube 335 may open.

### (E) Other Embodiments

Although some embodiments related to the present invention have been described heretofore, the present invention is not restricted to the abovementioned embodiments. Other embodiments will be described below. As a matter of course, the present invention, without being restricted even to the embodiments described below, appropriate modifications within the scope of technical ideas of the present invention are possible. Moreover, aforementioned embodiments and the embodiments mentioned below can be combined appropriately.

### (E-a) Other Embodiments of Injection Head

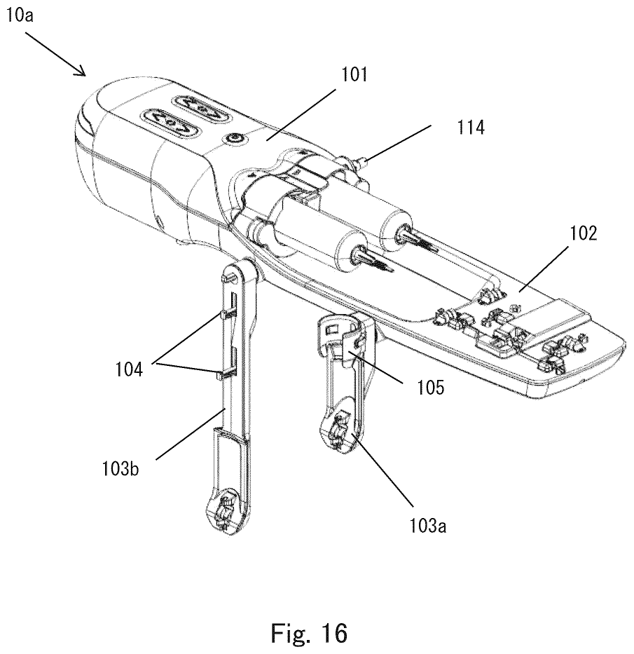

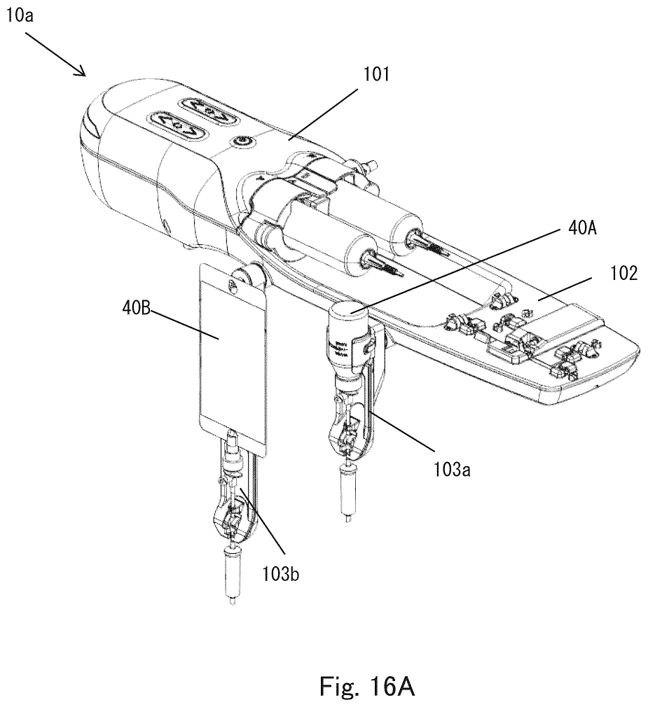

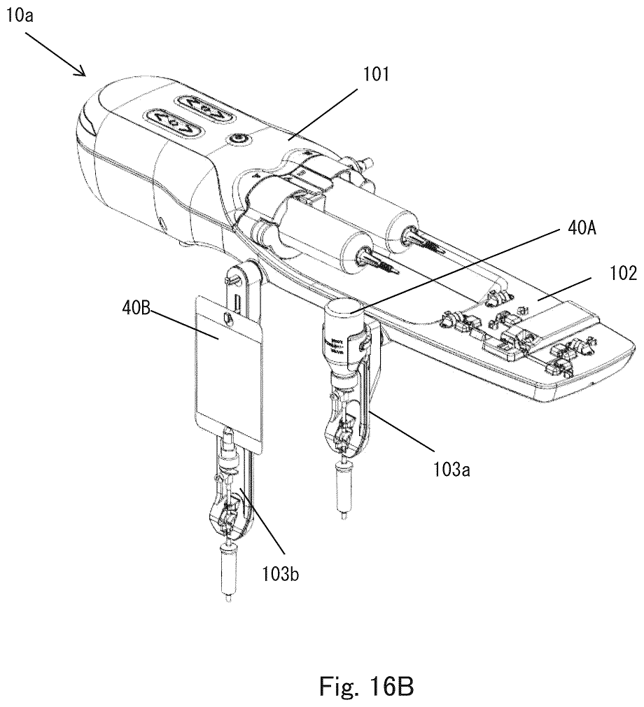

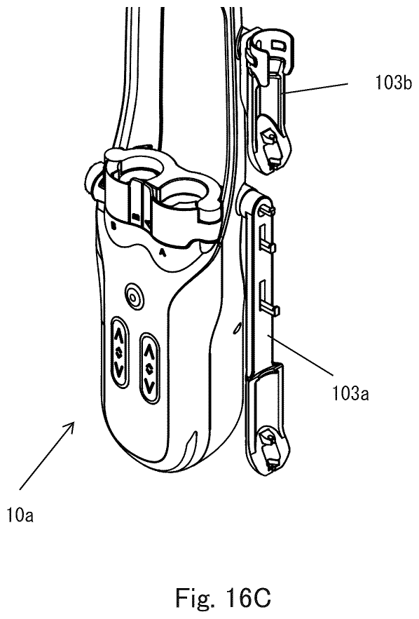

In FIG. 16, a perspective view of another embodiment of an injection head is shown. The injection head 10a shown in FIG. 16, similarly as the aforementioned injection head, also has a head body 101 on which the syringe is loaded, and the chemical-liquid circuit operating unit 102, and is pivotably supported by an appropriate stand etc. with the support shaft 114 as a center. However, in the present embodiment, a point that two chemical-liquid container holders 103a and 103b are supported by the chemical-liquid circuit operating unit 102 differs from the aforementioned embodiment. The first chemical-liquid container holder 103a is suitable for holding a bottle as the chemical-liquid container, and has a bottle receiver 105. A contrast medium can be cited as an example of a chemical liquid to be filled in the bottle. The second chemical-liquid container holder 103b is suitable for holding a bag as the chemical-liquid container, and has a plurality of hooks 104 for hanging the bag. The hooks 104 are disposed at different positions in a vertical direction, and it is preferable that each hook is of a folding type so that when a bag is hanged from the hook 104 at an upper side, the lower hook 104 does not hinder the hanging of the bag. A physiological saline solution can be cited as an example of the chemical liquid filled in the bag. In FIG. 16A, a state in which the first container 40A which is a bottle filled with contrast medium is held by the first chemical-liquid container holder 103a and the second container 40B which is a large-capacity bag is held by the second chemical-liquid container holder 103b is shown. In FIG. 16B, a state in which the first container 40A which is a bottled filled with contrast medium is held by the first chemical-liquid container holder 103a and the second container 40B which is a small-capacity bag is held by the second chemical-liquid container holder 103b is shown.

Moreover, it is preferable that the chemical-liquid container holders 103a and 103b are pivotably supported by a shaft parallel to the support shaft 114. Accordingly, it is possible to maintain a posture of the chemical-liquid container holders 103a and 103b even when a posture of the injection head 10a is changed such as the injection head 10a assuming an upward posture as shown in FIG. 16C. In this case, a spacing for attaching the chemical-liquid container holders 103a and 103b to the injection head 10a is designed such that even when the posture of the injection head 10a is changed, the chemical-liquid container holders 103a and 103b do not interfere mutually.

### (E-b) 2-Console System

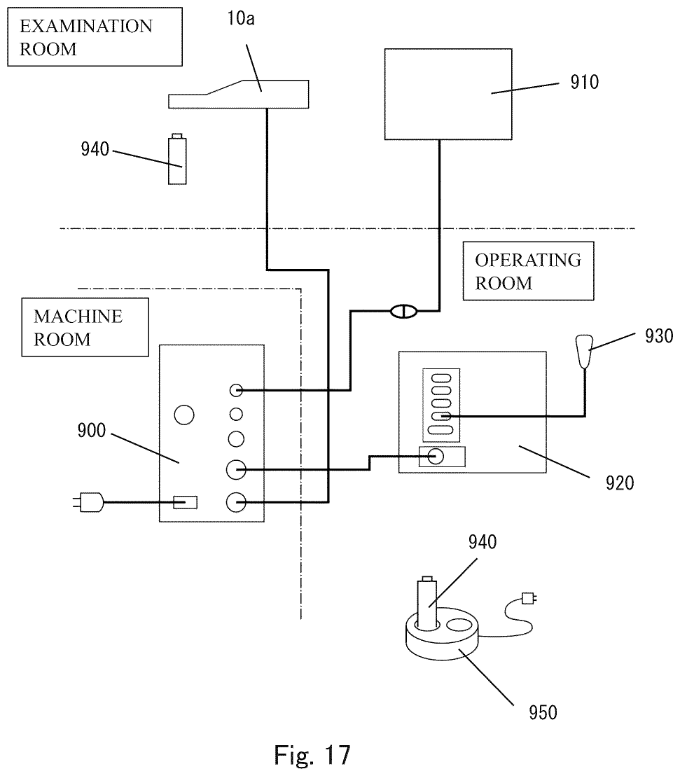

The chemical-liquid injector can have a plurality of consoles. In FIG. 17, a system configuration of a chemical-liquid injector having two consoles is shown. The chemical-liquid injector of the present embodiment has the injection head 10a, a main unit 900, a main console 910, a sub console 920, a variable hand switch 940, and an electrical charging stand 950. Moreover, the chemical-liquid injector has a hand switch 930 as an option. The system shown in FIG. 1 can also have the option.

The main unit 900 is provided with an electrical power supply unit which supplies electric power to the injection head 10a, the main console 910, and the sub console 920. Each of the main console 910 and the sub console 920 have a configuration and function similar to those of the console 10b shown in FIG. 1. The injection head 10a and the main console 910 are disposed in an examination room, and the sub console 920 is disposed in an operating room, and the main unit 900 is disposed in a machine room.

The hand switch 930 is one of input devices externally connected to the sub console 920, and is provided with a start button for starting an operation of injecting the chemical liquid. The chemical liquid is injected only while the start button is being pressed. The variable hand switch 940 is one of input devices wirelessly connected to the main console 910 to enable to inject the chemical liquid arbitrarily by an operation by the user in the examination room. The variable hand switch has a built-in rechargeable battery, and recharging of battery is possible by the electrical charging stand 950. Moreover, in the present embodiment, two variable hand switches 940 are provided including a spare one for a case in which the battery level becomes low. Communication standards for communication between the variable hand switch 940 and the main console 910 are not restricted, and for example, Bluetooth (registered trademark) standard can be used.

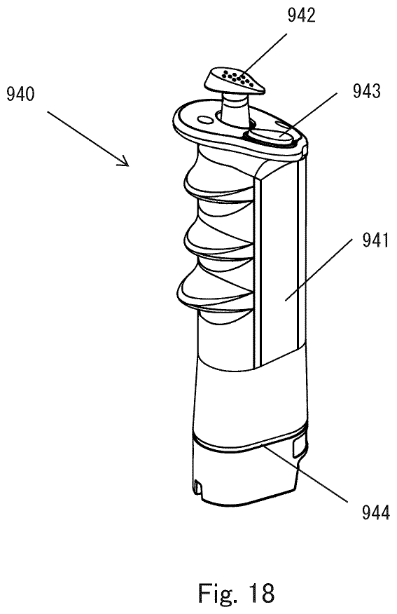

The variable hand switch 940 will be described below by referring to FIG. 18. The variable hand switch 940 has a hand switch body 941 with a grip, a first start button 942, a second start button 943, and an LED lamp 944 which is disposed on a side surface of the hand switch body 941. Moreover, a power-supply switch and a connector for recharging are provided on a bottom surface of the variable hand switch 940.

The first start button 942 is a button for starting an operation of injecting a first chemical liquid (for example, contrast medium), and the chemical liquid is injected only while the button is being pressed. The ‘first chemical liquid’ here includes a first chemical liquid which is diluted by a second chemical liquid (for example, physiological saline solution). Moreover, the first start button 942 is also capable of changing an injection rate of the chemical liquid according to a depth of button press, and injecting the chemical liquid at a constant injection rate regardless of the depth of button press. For instance, in cardio mode, the more the depth of button press of the first start button 942, the higher is the injection rate with which the chemical liquid is injected, and in angio mode, the chemical liquid is injected at a constant injection rate irrespective of the depth of button press of the first start button 942. The second start button 943 is a button for starting an operation of injecting the second chemical liquid. As injection modes by the second start button 943, there are two modes, which are, a first mode in which, the second chemical liquid is injected only while the second start button 943 is being pressed and a second mode in which, a predetermined volume of the second chemical liquid is injected when the second start button 943 is pressed. The user can set in advance as to by which mode to inject. The first start button 942 and the second start button 943 are disposed on an upper surface of the band switch body 941, and an arrangement is made such that the user, in a state of gripping the switch body 941, is capable of operating the first start button 942 and the second start button 943 by thumb.

Emission of light by the LED lamp 944 is controlled according to a state of the variable hand switch 940. For instance, orange color blinks during charging, and orange color is lit when the charging is over. Moreover, when the communication is not connected, blue color blinks, and in a state of the communication connected, blue color is lit.

The variable hand switch 940 and the main console 910 may be connected by a wired connection. In this case, an arrangement for a battery and charging are not included.

The variable hand switch 940 may have the first start button 942, the second start button 943, and furthermore, a third start button. In the existing situation, in the angio mode, after a catheter is inserted into a patient, for verifying a front-end portion of the catheter inserted, a small volume of contrast medium is discharged by using a foot switch, and while verifying the front-end position of the catheter, a front end of the catheter is guided to a target position. However, the foot switch being large in size, and being disposed on a floor, there were cases in which, the foot switch itself became an obstacle while inserting the catheter, and the user could not insert it at a free position. Therefore, by imparting a function of discharging the contrast medium by the foot switch to the variable hand switch 940, the user is capable of guiding the catheter front end to the target position while verifying the catheter front end position at a free position.

### (E-c) User Interface

Here, user interfaces such as various screens to be displayed on a display device of the console (console 10b, main console 942, and sub console 943) of the chemical-liquid injector, sounds for calling attention of the user and for informing a status of the system to the user will be described below.

When an electric power supply to the chemical-liquid injector is put ON, a predetermined initializing operation is carried out, and thereafter, a home screen is displayed on the display device of the console. An injection-mode selection key, an injection result key, and an environment setting key are displayed on the home screen. The injection-mode selection key is a key for selecting the injection mode, the injection result key is a key for displaying injection result, and the environment setting key is a key for setting various environments.

By tapping the environment setting key, an environment setting screen is displayed, and selection screens such as date, beep sound, charging operation, flush, priming operation are displayed as items to be set on this environment setting screen. From these selection screens, the user can select as to which item to set.

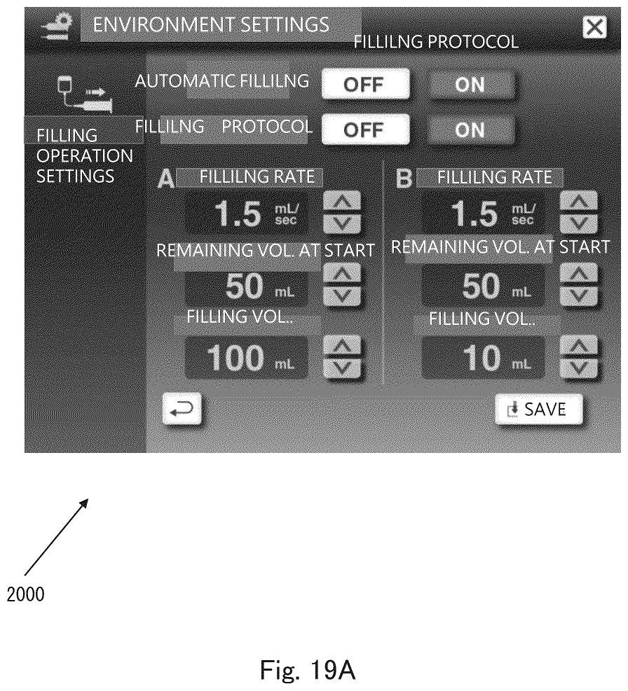

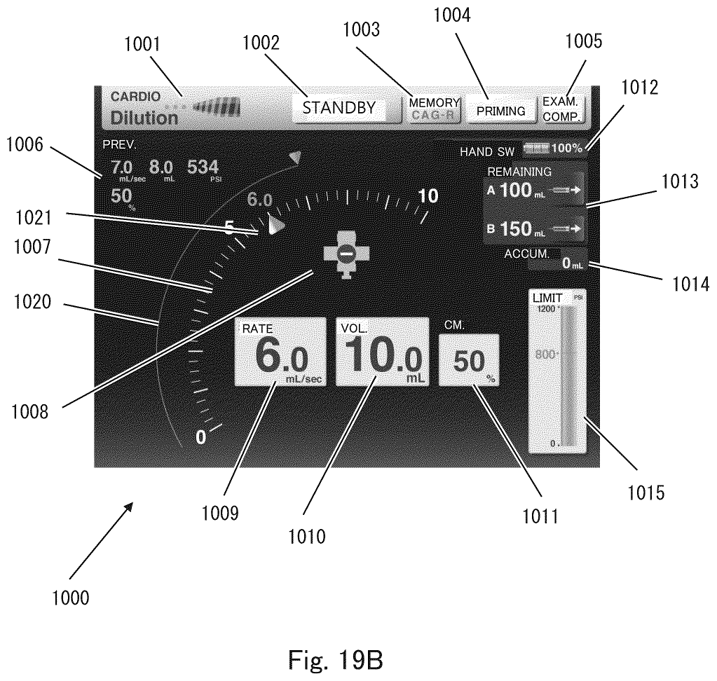

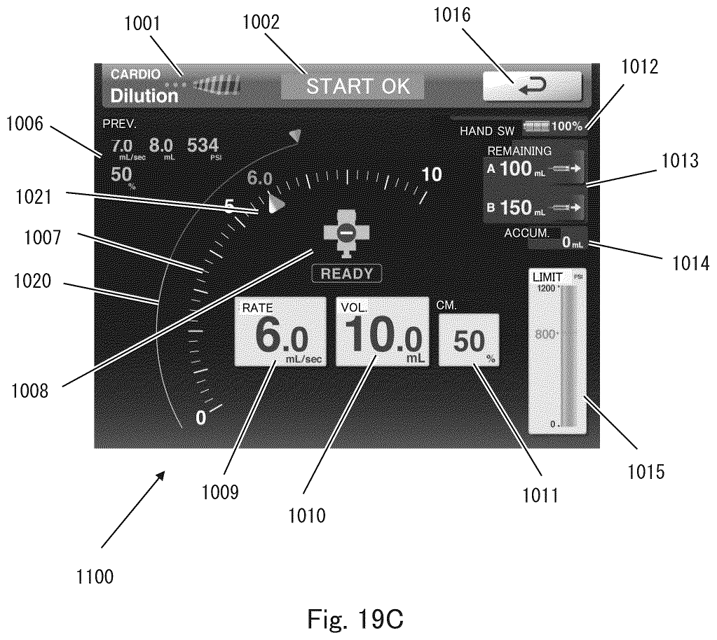

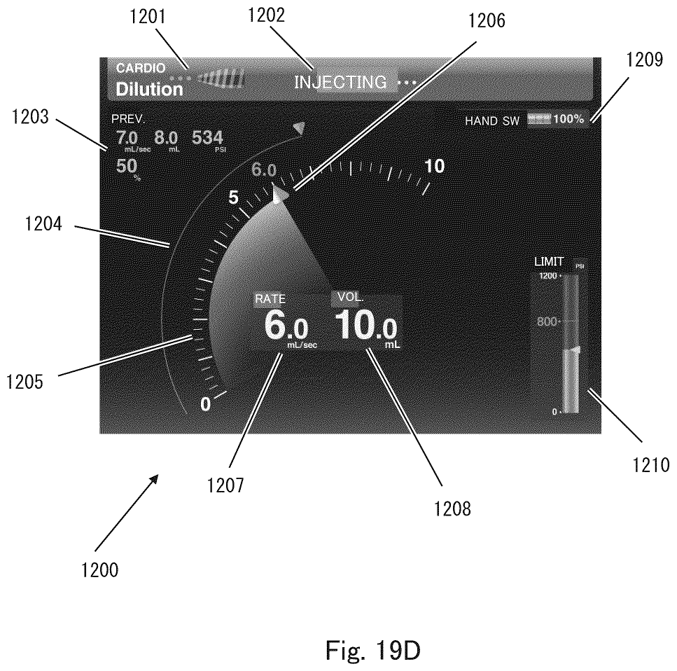

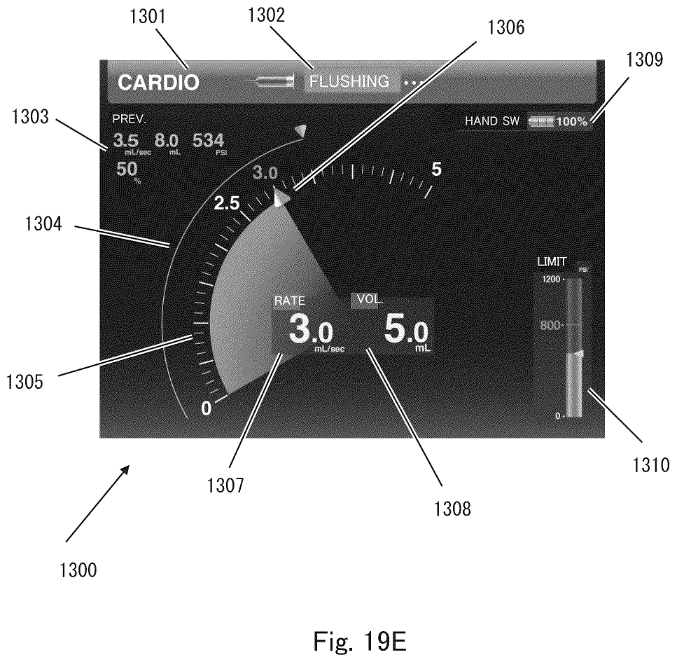

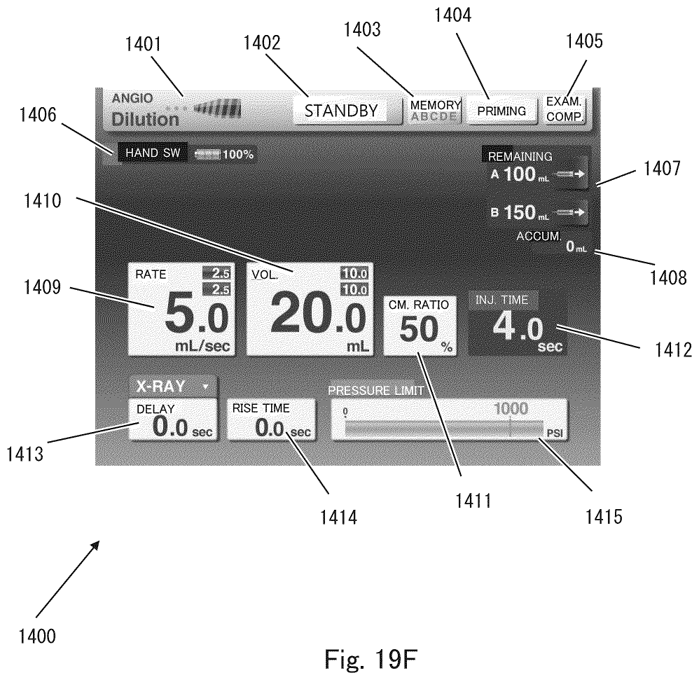

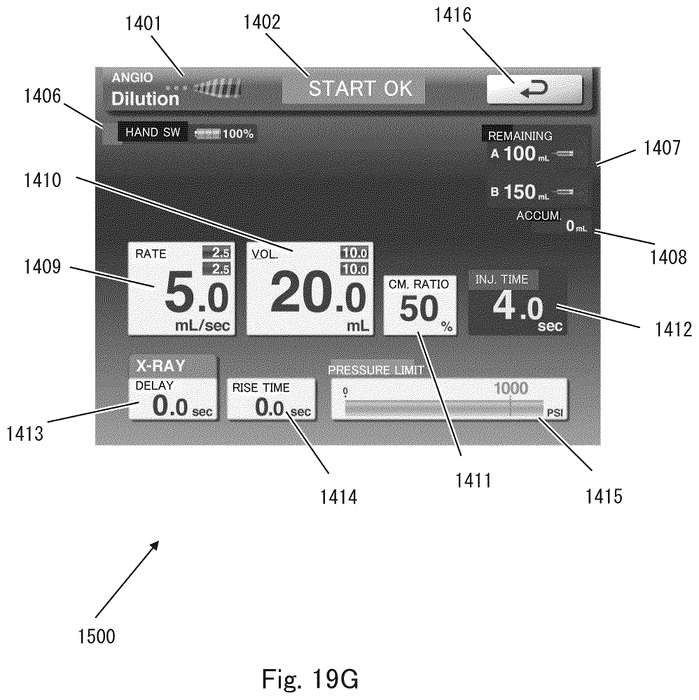

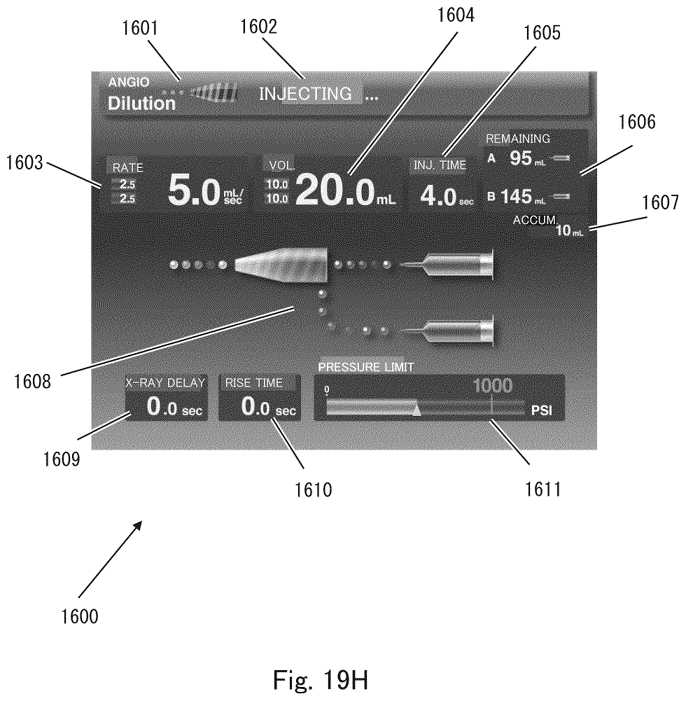

From among these, setting of filling operation for filling the chemical liquid into the syringe from the chemical-liquid container will be described below by referring to FIG. 19A which is a filling-operation setting screen displayed by selecting the filling operation on the selection screen. ON/OFF of filling operation, ON/OFF of filling protocol, filling rate, volume remaining at the start of filling, and filling volume of each of the chemical liquid A and chemical liquid B can be set on a filling-operation setting screen 2000.

Here, the filling protocol is a special protocol which reduces adhesion of air to an inner surface of the cylinder when the plunger is retracted for filling the chemical liquid in the cylinder. In this filling protocol, the chemical liquid is filled by carrying out in succession, a first phase of filling the chemical liquid at a first rate and a second phase of filling the chemical liquid at a rate higher than the first rate. A filling rate and a filling time in each of the first phase and the second phase are determined such that, a total of a volume filled in the first phase and a volume filled in the second phase is equivalent to a volume to be filled of the chemical liquid that has been set. The filling rate in the first phase and the second phase can be let to be ±1.5 mL/sec with respect to the filling rate in a case of carrying out the filling operation without changing the filling rate for example.

In a case in which the filling protocol is set to ON in the filling-operation setting screen 2000, the filling operation is carried out with the filling protocol described above. Whereas, in a case in which the filling protocol is set to OFF, the filling operation is carried out at a constant injection rate that has been set.

A user interface related to injection of chemical liquid will be described below.

In a case in which, the chemical-liquid injector is an injector to be used for an angio imaging system, the chemical-liquid injector can be configured to inject the contrast medium and physiological saline solution in an injection mode adapted to the two modes, the cardio mode and the angio mode. Particularly in these two modes, injecting the contrast medium upon diluting with physiological saline solution is also called as cardio dilution mode and angio dilution mode respectively.

FIG. 19B is an injection conditions setting screen 1000 in the cardio dilution mode. At an upper portion of this injection conditions setting screen 1000, a bar having an injection mode display 1001, a status display 1002, a memory key 1003, a priming key 1004, and an examination completion key 1005 is displayed. Below the bar, a previous examination information 1006, an injection rate display 1007 designed in a meter type, a status 1008 of flow channel opening/closing valve (display when closed is shown in the diagram), a rate key 1009, a volume key 1010, and a dilution key 1011. On a right side of the screen, a battery display 1012 of variable hand switch, a remaining volume display and filling key 1013, an accumulated volume display 1014, and a pressure limit key 1015 are displayed. Moreover, near the injection rate display 1007, a previous result rate mark 1020 and a set rate mark 1021 are displayed.

The priming key 1004 is a key for executing priming, and by the user tapping the priming key 1004, the screen changes to a priming screen (not shown in the diagram). After the completion of priming, it is possible to return to the injection conditions setting screen 1000 from the priming screen by a predetermined operation. Moreover, for the priming of transducer, it is possible to set priming conditions in advance as, delay time in a range of 0 sec to 10 sec, injection rate in a range of 0.1 mL/sec to 10 mL/sec, and injection volume in a range of 1 mL to 50 mL from the environment setting screen displayed after the power of the console is put on. The delay time is a grace time after the start of priming operation is carried out till the priming operation is performed, and it is possible to set according to time required for movement up to a location of transducer after the start operation is carried out by the user.

The examination completion key 1005 is a key for examination completion processing, and as the user taps the examination completion key 1005, a selection screen prompting the user to select whether to carry out the subsequent examination or to end the examination is popped up, and moves to the subsequent processing according to the result of selection by the user. The selection screen is also popped up when detaching of the single-time use section is detected.

The dilution key 1101 is a key for setting rate of dilution of the contrast agent by physiological saline solution. As the user taps the dilution key 1101, dilution rate setting screen is popped up, and it is possible to set the dilution rate arbitrarily on the dilution rate setting screen. The dilution rate setting screen may be for any method setting the dilution rate such as, a method of setting the dilution rate by directly inputting a value by tenkey, a method of setting the dilution rate by sliding a slider of a slide bar by swipe operation, and a method of setting the dilution rate by sliding by key operation. Moreover, the dilution rate may be expressed by any expression such as dilution proportion (for example, ‘XX %’), dilution ratio (for example, XX:ΔΔ), and multiplying factor (for example, XX times), and the user may select arbitrarily the expression for the dilution rate.

The battery display 1012 is a display for displaying a charging status (battery level) of a battery of the variable hand switch, and the battery level is displayed in units of 10% for example. Moreover, when the battery level has changed from 40% to 30%, from 30% to 20%, and from 20% to 10%, sound notifying the change in the battery level may be generated from each console, as well as by blinking of the battery display 1012. Furthermore, as the battery level becomes 30% for example, a warning message of ‘battery-level low’ may be displayed on the battery display 1012, and/or the color of display may change to orange color.

When the setting of injection conditions is completed and when the user taps the status display 1002, the screen changes from the injection conditions setting screen 1000 to a status OK screen 1100 shown in FIG. 19C, and the injection mode display 1001, the status display 1002, and a return key 1016 are displayed on a bar at an upper portion of the screen. By the user pressing a status button of the hand switch in this state. injection of the contrast medium and physiological saline solution starts. It is possible to return to the injection conditions setting screen 1000 by tapping of the return key 1016 by the user.

As the injecting starts, the screen changes to an injecting-in-progress screen 1200 shown in FIG. 19D. An injection mode display 1201, a status display 1202, a previous result information 1203, a previous result rate mark 1204, an injection rate display 1205 designed in a meter type, set rate mark 1206, an injection rate display 1207, an injection volume display 1208, a battery display 1209, and a pressure display 1210 are displayed on the injecting-in-progress screen 1200. In the injection rate display 1205, an animation display of the current injection rate is displayed. In the pressure display 1210, a pressure limit value that has been set and a pressure during injecting are displayed.

An arrangement may be made such that during the injecting operation, the user is notified that the injecting operation is in progress by generating an intermittent beep sound from the console. Moreover, including even a case of the injection rate changing with the lapse of time, an interval between the beep sound may be changed according to the injection rate, and a tone of the beep sound may be changed according to the chemical liquid injected (whether it is a chemical liquid on the A side or a chemical liquid on the B side). This can be applied similarly to the operation of the variable hand switch 940.

Moreover, during the injecting operation, as the injection pressure reaches a pressure limit value that has been set, a pressure limiter is activated, and this is notified to the user by at least one of a display of the pressure limit key 1015, notifying sound from the console, and other means. However, regarding the notifying sound, when other instrument generating the notifying sound is used simultaneously with the chemical-liquid injector, since there is a possibility that the notifying sound generated from the other instrument is not susceptible to be heard, an arrangement is made such that the user can set ON/OFF arbitrarily. ON/OFF of the notifying sound can be set from the aforementioned environment setting screen. However, at the time of angio dilution mode, the notifying sound is generated even when the notifying sound is set to OFF.

On the other hand, during the flushing by physiological saline solution, a flushing-in-progress screen shown in FIG. 19E is displayed. An injection mode display 1301, a status display 1302, a previous result information 1303, a previous result rate mark 1304, an injection rate display 1305 designed in a meter type, a set rate mark 1306, an injection rate display 1307, an injection volume display 1308, a battery display 1309, and an injection pressure display 1310 are displayed on a flushing-in-progress screen 1300

It is possible to set flushing conditions in advance from the environment setting screen as, injection rate in a range of 0.1 mL/sec to 5 mL/sec and injection volume in a range of 1 mL to 20 mL for example.

Next, change of screens in the angio dilution mode will be described below. FIG. 19F is an injection conditions setting screen 1400 in the angio dilution mode. At an upper portion of this injection conditions setting screen 1400, a bar having an injection mode display 1401, a status display 1402, a memory key 1403, a priming key 1404, and an examination completion key 1405 is displayed. Below the bar, a battery display 1406, a remaining volume display/filling key 1407, an accumulated volume display 1408, a rate key 1409, a volume key 1410, a contrast medium proportion key 1411, injecting time key 1412, a delay key 1413, a rise time key 1414, and a pressure limit key 1415 are displayed.

The rate key 1409 and the volume key 1410 are used for setting the injection rate and the injection volume respectively. Moreover, the rate and volume of each of the contrast medium and physiological saline solution are also displayed on the rate key 1409 and the volume key 1410 respectively. The contrast medium proportion key 1411 is used for setting a proportion of the contrast medium with respect to the injection volume. Since the contrast medium proportion key 1411 has a function similar to that of the aforementioned dilution key 1011 (refer to FIG. 10B), the description thereof will be omitted here. An injecting time is displayed on the injecting time key 1412, The delay key 1413 is used for setting a delay format and delay time. The rise time key 1414 is used for setting a time to be taken for reaching the injection rate that has been set. The pressure limit key 1415 is used for setting a limit value of injection pressure.

When the setting of injection conditions is completed and when the user taps the status display key 1402, the screen changes from the injection conditions setting screen 1400 to a status OK screen 1500 shown in FIG. 19G, and the injection mode display 1401, the status display 1402, and a return key 1416 are displayed on a bar at an upper portion of the screen. Injection of the contrast medium and physiological saline solution starts by pressing of a start button of the hand switch in this state by the user. It is possible to return to the injection conditions setting screen 1400 by tapping of the return key 1016 by the user.

As the injecting starts, the screen changes to an injecting-in-progress screen 1600 shown in FIG. 19H. An injection mode display 1601, a status display 1602, a rate display 1603, a volume display 1604, an injecting time display 1605, a remaining volume display 1606, an accumulated volume display 1607, a remaining volume animation display 1608, a delay display 1609, a rise time display 1610, an injecting pressure display 1611 are displayed on the injecting-in-progress screen 1600. A remaining volume of the chemical liquid in the syringe and a state in which the chemical liquid is injected are displayed by animation display on the remaining volume animation display 1608.

In both of the cardio mode and the angio mode, the injection result can be verified from the injection result screen. The injection result includes date, starting time, total volume injected, number of cardio injections, and number of angio injections. Regarding the number of angio injections, details other than mentioned above, such as injection pattern, maximum rate, injected volume, maximum pressure, delay time, and rise time can be verified.

### Description of Reference Numerals

10 chemical-liquid injector
10a injection head
10b console
22 syringe
22a flange
22b nozzle portion
22c syringe protrusion
30 chemical-liquid circuit
101 head body
102 chemical-liquid circuit operating unit
103, 103a, 103b chemical-liquid container holder
111 clamper
112 presser
113 operating section
120 syringe drive mechanism
121 front frame
122 rear frame
123 side frame
124 ball screw mechanism
125 linear guide
126 slider
127 ram
301a first main line
301b first sub line
302a second main line
302b second sub line
303 patient line
304 transducer line
360 syringe connector
360a connector protrusion
400 suction tube unit
410 tube body
420 suction valve
421 first case
422 second case
423 valve element
424 coil spring
430 opening/closing dust cap
440 spike
440a infusing flow channel
440b piercing portion
440d air vent
441 filter
501 housing
501a, 501b conduit portions
502 piston
502a head
503 stopper structure
503a protrusion
503b recess
506 sealing ring
610, 620 unidirectional valve
610a valve chest
611, 621 first case
611a receiving recess
611b, 621b downstream-side flow channel
611c valve-element position regulating mechanism
612, 622 second case
612a valve seat
612b, 622b upstream-side flow channel
613, 623 valve element
624 gasket
625 bias applying spring
710, 780 air sensor
720, 730 flattening mechanism
740 flow channel opening/closing valve drive mechanism
841 air sensor
900 main unit
910 main console
920 sub console
930 hand switch
940 variable hand switch
950 electrical charging stand

1. A chemical-liquid injector on which a chemical-liquid circuit is detachably mounted, comprising:
a chemical-liquid circuit mounting section on which the chemical-liquid circuit having a patient line and a transducer line which is branched from the patient line, as flow channels of a chemical liquid, is mounted; and
a flow channel opening/closing mechanism which is at a downstream side of a branching portion of the patient line and the transducer line, of the chemical-liquid circuit mounting section.

2. The chemical-liquid injector according to claim 1, wherein
the patient line has a flow channel opening/closing valve at a downstream side of a branching portion with the transducer line, and
the flow channel opening/closing mechanism is a flow channel opening/closing valve drive mechanism which opens and closes the flow channel opening/closing valve.

3. The chemical-liquid injector according to claim 1, wherein
the patient line has a tube at a downstream side of a branching portion with the transducer line, and
the flow channel opening/closing mechanism is a flattening mechanism which flattens the tube.
